(19) United States
(12) Patent Application Publication
(10) Pub. No.: US 2026/0234167 A1
(43) Pub. Date: Aug. 13, 2026

(54) SOLID STATE FORMS OF DELGOCITINIB AND PROCESS THEREOF

(71) Applicant: ASSIA CHEMICAL INDUSTRIES LTD., Tel Aviv (IL)

(72) Inventors: Anantha Rajmohan Muthusamy, Sivakasi (IN); Amit Singh, Greater Noida (IN); Prathap Rengaraj, Madurai (IN)

(21) Appl. No.: 19/157,498

(22) PCT Filed: Feb. 28, 2024
(86) PCT No.: PCT/IB2024/051909

§ 371 Date: Aug. 18, 2025

(30) Foreign Application Priority Data
Feb. 28, 2023 (IN) 202311013369
Mar. 16, 2023 (IN) 202311017861
May 30, 2023 (IN) 202311037163

(51) Int. Cl. C07D 519/00; A61K 31/519

(52) U.S. Cl. CPC C07D 519/00; A61K 31/519

(57) ABSTRACT

The present disclosure relates to Delgocitinib solid state forms, processes for preparation thereof, pharmaceutical compositions thereof, and methods of use thereof.

### FIELD OF THE INVENTION

The present disclosure relates to Delgocitinib solid state forms, in embodiments crystalline polymorphs or salts of Delgocitinib, processes for preparation thereof, pharmaceutical compositions thereof, and methods of use thereof.

### BACKGROUND

Delgocitinib (JTE-052) which has the chemical name 3-[(3S,4R)-3-methyl-7-(7H-pyrrolo[2,3-d]pyrimidin-4-yl)-1,7-diazaspiro[3.4]octan-1-yl]-3-oxopropanenitrile, is a pan-Janus kinase (JAK) inhibitor and is approved for treatment of atopic dermatitis (AD) in Japan. As described in U.S. Pat. No. 8,609,647, Delgocitinib has the following chemical structure:

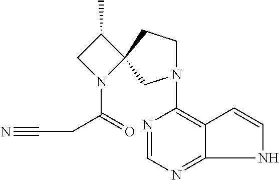 [SMILES: C[C@H]1CN(C(=O)CC#N)[C@]12CCN(c1ncnc3[nH]ccc13)C2]

Delgocitinib preparation is disclosed in U.S. Pat. Nos. 10,822,354 and 8,609,647 and Delgocitinib polymorphs are described in U.S. Pat. Nos. 11,339,181, and 11,312,728.

Polymorphism, the occurrence of different crystal forms, is a property of some molecules and molecular complexes. A single compound, like Delgocitinib, may give rise to a variety of polymorphs having distinct crystal structures and physical properties like melting point, thermal behaviors (e.g. measured by thermogravimetric analysis—“TGA”, or differential scanning calorimetry—“DSC”), X-ray powder diffraction (XRPD) pattern, infrared absorption fingerprint, Raman absorption fingerprint, and solid state (¹³C-)NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms may provide a basis for improving formulation, for example, by facilitating better processing or handling characteristics, improving the dissolution profile, or improving stability (polymorph as well as chemical stability) and shelf-life. These variations in the properties of different salts and solid state forms may also provide improvements to the final dosage form, for instance, if they serve to improve bioavailability. Different salts and solid state forms of an active pharmaceutical ingredient may also give rise to a variety of polymorphs or crystalline forms, which may in turn provide additional opportunities to use variations in the properties and characteristics of a solid active pharmaceutical ingredient for providing an improved product.

Discovering new salts and solid state forms of a pharmaceutical product can provide materials having desirable processing properties, such as ease of handling, ease of processing, storage stability, and ease of purification or as desirable intermediate crystal forms that facilitate conversion to other salts or polymorphic forms. New polymorphic forms and new salts of a pharmaceutically useful compound can also provide an opportunity to improve the performance characteristics of a pharmaceutical product (dissolution profile, bioavailability, etc.). It enlarges the repertoire of materials that a formulation scientist has available for formulation optimization, for example by providing a product with different properties, e.g., a different crystal habit, higher crystallinity or polymorphic stability which may offer better processing or handling characteristics, improved dissolution profile, or improved shelf-life. For at least these reasons, there is a need for additional salts and solid state forms (including solvated forms) of Delgocitinib.

### SUMMARY OF THE INVENTION

The present disclosure relates to Delgocitinib solid state forms, crystalline polymorphs thereof or amorphous form, to processes for preparation thereof, and to pharmaceutical compositions comprising solid state form thereof.

In particular, the present disclosure provides crystalline forms of Delgocitinib designated as Forms DL1-DL13 and crystalline Delgocitinib hydrochloride (defined herein).

The present disclosure further provides process for preparing Delgocitinib and solid state forms or crystalline polymorphs thereof.

In another aspect, the present disclosure encompasses the above described solid state forms or crystalline polymorphs of Delgocitinib and crystalline Delgocitinib hydrochloride for use in the preparation of pharmaceutical compositions and/or formulations, preferably for use in medicine, preferably for treating atopic dermatitis (AD).

In another aspect, the present disclosure encompasses the use of any one of the above described solid state forms or crystalline polymorphs of Delgocitinib and Delgocitinib hydrochloride for the preparation of pharmaceutical compositions and/or formulations, preferably for use in medicine, preferably for treating atopic dermatitis (AD). In yet another embodiment, the present disclosure encompasses pharmaceutical compositions comprising any one of the solid state forms or crystalline polymorphs of Delgocitinib and Delgocitinib hydrochloride.

In a specific embodiment, the present disclosure encompasses pharmaceutical formulations comprising any one of the above described solid state forms or crystalline polymorphs of Delgocitinib and Delgocitinib hydrochloride, and at least one pharmaceutically acceptable excipient.

The present disclosure further encompasses processes to prepare said pharmaceutical formulations of Delgocitinib and Delgocitinib hydrochloride, comprising combining any one of the above described solid state forms or crystalline polymorphs of Delgocitinib and Delgocitinib hydrochloride, or pharmaceutical compositions comprising it, and at least one pharmaceutically acceptable excipient.

The solid state forms or crystalline polymorphs of Delgocitinib and Delgocitinib hydrochloride as defined herein as well as the pharmaceutical compositions or formulations comprising it can be used as medicaments, particularly for treating complement mediated diseases, preferably for the treatment of atopic dermatitis, comprising administering a therapeutically effective amount of the solid state form of the present disclosure, or at least one of the above pharmaceutical compositions or formulations, to a subject suffering from atopic dermatitis, or otherwise in need of the treatment.

The present disclosure also provides methods of treating atopic dermatitis, comprising administering a therapeutically effective amount of any one of the solid state forms or crystalline polymorphs of Delgocitinib and Delgocitinib hydrochloride of the present disclosure, or at least one of the above pharmaceutical compositions or formulations, to a subject suffering from atopic dermatitis, or otherwise in need of the treatment.

The present disclosure also provides the uses of the solid state forms or crystalline polymorphs of Delgocitinib and Delgocitinib hydrochloride of the present disclosure, or at least one of the above pharmaceutical compositions or formulations, for the manufacture of medicaments for treating atopic dermatitis.

### BRIEF DESCRIPTION OF THE DRAWINGS

FIG. 1 shows an X-ray powder diffraction (XRPD) pattern for crystalline Delgocitinib Form DL1.

FIG. 2 shows an XRPD pattern of crystalline Delgocitinib Form DL2.

FIG. 3 shows an XRPD pattern of crystalline Delgocitinib Form DL3.

FIG. 4 shows an XRPD pattern of crystalline Delgocitinib Form DL4.

FIG. 5 shows an XRPD pattern of Amorphous Delgocitinib.

FIG. 6 shows an XRPD pattern of crystalline Delgocitinib Form DL5.

FIG. 7 shows an XRPD pattern of crystalline Delgocitinib Form DL6.

FIG. 8 shows an XRPD pattern of crystalline Delgocitinib Form DL7.

FIG. 9 shows an XRPD pattern of crystalline Delgocitinib Form DL8.

FIG. 10 shows an XRPD pattern of crystalline Delgocitinib Form DL9.

FIG. 11 shows an XRPD pattern of crystalline Delgocitinib Form DL10.

FIG. 12 shows an XRPD pattern of crystalline Delgocitinib Form DL11.

FIG. 13 shows an XRPD pattern of crystalline Delgocitinib Form DL12.

FIG. 14 shows an XRPD pattern of crystalline Delgocitinib Form DL13.

FIG. 15 shows an XRPD pattern of crystalline Delgocitinib hydrochloride.

FIG. 16 shows an XRPD pattern of crystalline Delgocitinib Form Alpha.

### DETAILED DESCRIPTION OF ILLUSTRATIVE EMBODIMENTS

The present disclosure relates to solid state forms or crystalline polymorphs of Delgocitinib and Delgocitinib hydrochloride, to processes for preparation thereof and to pharmaceutical compositions comprising at least one of, or combination of these solid state forms. In particular, the present disclosure relates to solid state forms of Delgocitinib designated as Form DL1-Form DL13 and Delgocitinib hydrochloride (defined herein).

The Delgocitinib solid state forms, according to the present disclosure may have advantageous properties selected from at least one of: chemical or polymorphic purity, flowability, solubility, dissolution rate, bioavailability, morphology or crystal habit, stability-such as chemical stability as well as thermal and mechanical stability with respect to polymorphic conversion, stability towards dehydration and/or storage stability, a lower degree of hygroscopicity, low content of residual solvents, adhesive tendencies and advantageous processing and handling characteristics such as compressibility, and bulk density.

A crystal form may be referred to herein as being characterized by graphical data “as depicted in” a Figure. Such data include, for example, powder X-ray diffractograms and solid state NMR spectra. As is well-known in the art, the graphical data potentially provides additional technical information to further define the respective solid state form (a so-called “fingerprint”) which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms.

A crystal form of Delgocitinib, referred to herein as being characterized by graphical data “as depicted in” a Figure will thus be understood to include any crystal form of Delgocitinib characterized with the graphical data having such small variations, as are well known to the skilled person, in comparison with the Figure.

The solid state forms or crystalline polymorphs of Delgocitinib and Delgocitinib hydrochloride as described in any aspect or embodiment of the present disclosure may be polymorphically pure, or substantially free of any other solid state forms of Delgocitinib and Delgocitinib hydrochloride.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% (w/w) of any other forms of the subject compound as measured, for example, by XRPD. Thus, solid states of Delgocitinib or Delgocitinib hydrochloride described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject solid state form of Delgocitinib or Delgocitinib hydrochloride. Accordingly, in some embodiments of the disclosure, the described solid state form of Delgocitinib and Delgocitinib hydrochloride may contain from about 1% to about 20% (w/w), from about 5% to about 20% (w/w), or from about 5% to about 10% (w/w) of one or more solid state forms of Delgocitinib and Delgocitinib hydrochloride.

The solid state forms or crystalline polymorphs of Delgocitinib and Delgocitinib hydrochloride as described in any aspect or embodiment of the present disclosure may be chemically pure, or substantially free of any other compounds.

A compound may be referred to herein as chemically pure or purified compound or as substantially free of any other compounds. As used herein, the terms “chemically pure” or “purified” or “substantially free of any other compounds” refer to a compound that is substantially free of any impurities including enantiomers of the subject compound, diastereomers or other isomers. A chemically pure or purified compound or a compound that is substantially free of any other compound will be understood to mean that it contains about 10% (w/w) or less, about 5% (w/w) or less, about 4% (w/w) or less, about 3% (w/w) or less, about 2% (w/w) or less, about 1.5% (w/w) or less, about 1% (w/w), about 0.8% (w/w) or less, about 0.6% (w/w) or less about 0.4% (w/w) or less about 0.2% (w/w) or less or less, about 0.1% (w/w) or less or about 0% of any other compound as measured, for example, by HPLC. Alternatively, A chemically pure or purified compound or a compound that is substantially free of any other compound will be understood to mean that it contains about 10% area percent or less, about 5% area percent or less, about 4% area percent or less, about 3% area percent or less, about 2% area percent or less, about 1.5% area percent or less, about 1% area percent or less, about 0.8% area percent or less, about 0.6% area percent or less, about 0.4% area percent or less, about 0.2% area percent or less, about 0.1% area percent or less, or about 0% of any other compound as measured by HPLC. Thus, pure or purified Delgocitinib and Delgocitinib hydrochloride described herein as substantially free of any compounds would be understood to contain greater than about 90% (w/w), greater than about 95% (w/w), greater than about 96% (w/w), greater than about 97% (w/w), greater than about 98% (w/w), greater than about 98.5% (w/w), greater than about 99% (w/w), greater than about 99.2%, (w/w) greater than about 99.4% (w/w), greater than about 99.6% (w/w), greater than about 99.8% (w/w), greater than about 99.9% (w/w), or about 100% of the subject Delgocitinib and Delgocitinib hydrochloride. Alternatively, pure or purified Delgocitinib and Delgocitinib hydrochloride described herein as substantially free of any compounds would be understood to contain greater than about 90% area percent, greater than about 95% area percent, greater than about 96% area percent, greater than about 97% area percent, greater than about 98% area percent, greater than about 98.5% area percent, greater than about 99% area percent, greater than about 99.2%, area percent, greater than about 99.4% area percent, greater than about 99.6% area percent, greater than about 99.8% area percent, greater than about 99.9% area percent, or about 100% of the subject Delgocitinib and Delgocitinib hydrochloride.

As used herein, unless stated otherwise, XRPD peaks reported herein are preferably measured using CuKα radiation, λ=1.5418 Å, preferably, XRPD peaks reported herein are measured using Cukα radiation, λ=1.5418 Å, at a temperature of 25±3° C.

As used herein, the term “isolated” in reference to solid state form of Delgocitinib and Delgocitinib hydrochloride of the present disclosure corresponds to solid state form of Delgocitinib and Delgocitinib hydrochloride that is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

As used herein, and unless stated otherwise, the term “anhydrous” in relation to crystalline Delgocitinib and Delgocitinib hydrochloride which does not include any crystalline water (or other solvents) in a defined, stoichiometric amount within the crystal. Moreover, an “anhydrous” form does not contain more than about 1% (w/w) of either water or organic solvents as measured for example by TGA, Karl Fischer or by other suitable technique.

The term “solvate”, as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

The amount of solvent employed in a chemical process, e.g., a reaction or crystallization may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding (methyl tert-butyl ether) MTBE (1.5 v/v) to a 100 ml reaction mixture would indicate that 150 mL of MTBE was added.

As used herein the term non-hygroscopic in relation to crystalline Delgocitinib and Delgocitinib hydrochloride, refers to less than about 1.0% (w/w) absorption of water at about 25° C. and about 80% relative humidity (RH), as determined for example by TGA or other suitable technique.

As used herein, the term “reduced pressure” refers to a pressure of about 10 mbar to about 500 mbar.

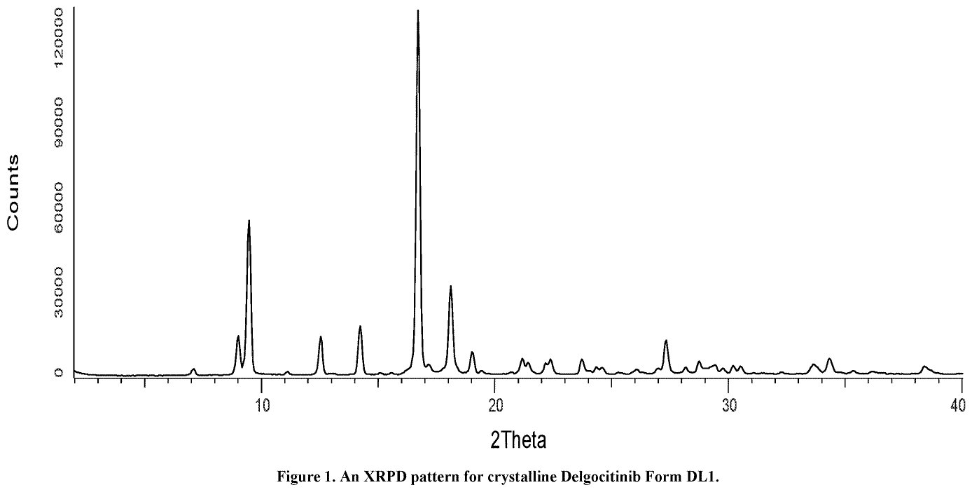

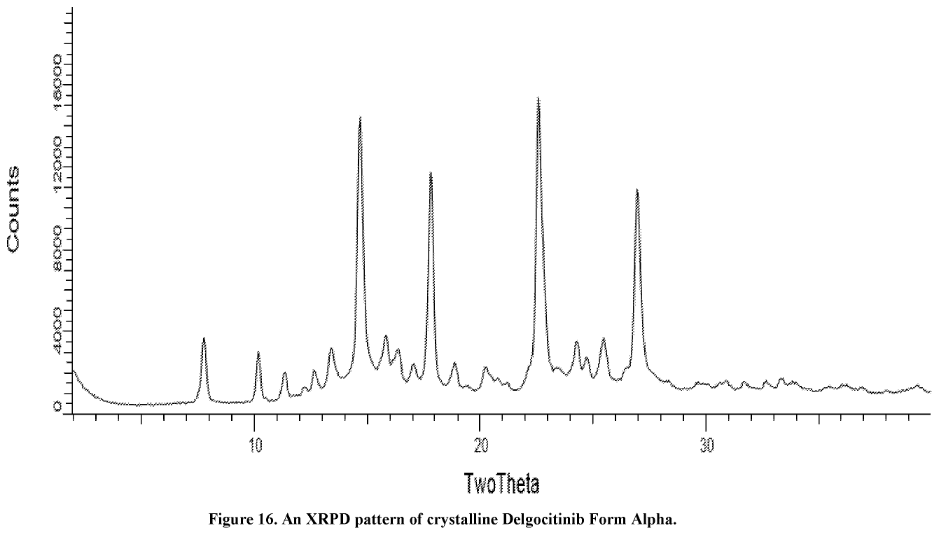

As used herein, Delgocitinib Form Alpha, is Delgocitinib Form alpha (“Form a”) described in U.S. Pat. No. 11,339,181 by XRPD (FIG. 1) and by XRPD pattern described herein (FIG. 16). Form Alpha can be prepared following example 1 described in U.S. Pat. No. 11,339,181 and by example 19 described below.

As used herein, and unless indicated otherwise, the term “thermo-dynamical stability” in relation to solid state forms or crystalline polymorphs of Delgocitinib and Delgocitinib hydrochloride refers to resistance of the solid state form or crystalline polymorph to polymorphic conversion under certain conditions, for example, heating, melting or dissolving. In some embodiments, the term refers to less than about 20% (w/w), about 10% (w/w), about 5% (w/w), about 1% (w/w), about 0.5% (w/w), or about 0% (w/w) conversion of crystalline Delgocitinib and Delgocitinib hydrochloride to any other solid state form of Delgocitinib and Delgocitinib hydrochloride as measured by XRPD. In some embodiments, the conversion is about 1% (w/w) to about 20% (w/w), about 1% (w/w) to about 10% (w/w) or about 1% (w/w) to about 5% (w/w).

The present disclosure comprises a crystalline Delgocitinib designated as Form DL1. Delgocitinib crystalline Form DL1 can be characterized by data selected from one or more of the following: an XRPD pattern having peaks at 9.5, 12.6, 14.3, 16.8 and 18.1 degrees 2-theta±0.2 degrees 2-theta; an XRPD pattern substantially as depicted in FIG. 1; or combinations of these data.

Crystalline Delgocitinib Form DL1 may be further characterized by data selected from one or more of the following: an XRPD pattern having peaks at 9.5, 12.6, 14.3, 16.8 and 18.1 degrees two theta±0.2 degrees two theta; and also having one, two or three additional peaks selected from 9.0, 19.1 and 27.4 degrees two theta±0.2 degrees two theta; or combinations of these data.

Crystalline Delgocitinib Form DL1 may alternatively be characterized by XRPD pattern having peaks at 9.0, 9.5, 12.6, 14.3, 16.8, 18.1, 19.1 and 27.4 degrees two theta±0.2 degrees two theta.

Crystalline Delgocitinib Form DL1 as described in any aspect or embodiment disclosed herein may be additionally characterized by an XRPD pattern having an absence of peaks at any one, two, three, four, five, six, or seven of: 3.0 to 6.5 degrees two theta±0.2 degrees two theta; 7.8 to 8.5 degrees two theta±0.2 degrees two theta; 10.0 to 10.6 degrees two theta±0.2 degrees two theta; 11.6 to 12.0 degrees two theta±0.2 degrees two theta; 13.6 to 13.8 degrees two theta±0.2 degrees two theta; 19.9 to 20.2 degrees two theta±0.2 degrees two theta; and 22.9 to 23.4 degrees two theta±0.2 degrees two theta.

Crystalline Delgocitinib Form DL1 may be characterized by each of the above characteristics alone/or by all possible combinations, e.g. by XRPD pattern having peaks at 9.5, 12.6, 14.3, 16.8 and 18.1 degrees two theta±0.2 degrees two theta and an XRPD pattern as depicted in FIG. 1, and combinations thereof.

Crystalline Delgocitinib Form DL1 may be characterized as a hydrate, preferably monohydrate.

The above Delgocitinib Form DL1 as described in any aspect or embodiment of the present disclosure can be prepared by a process including suspending Delgocitinib (preferably Form alpha, Form DL7 or Form DL3 as described in any aspect or embodiment of the disclosure) in water, preferably at a temperature of: about 30° C. to about 70° C., about 40° C. to about 65° C., about 50° C. to about 65° C., about 55° C. to about 65° C., or about 60° C., optionally with stirring; optionally isolating the Delgocitinib Form DL1, and optionally drying.

According to any aspect or embodiment of the process, water may be used in an amount of: about 20 to about 40 vol, about 25 to about 35 vol, and in some embodiments about 30 vol. The mixture may be heated to a temperature of: about 30° C. to about 70° C., about 40° C. to about 65° C., about 50° C. to about 65° C., about 55° C. to about 65° C., or about 60° C., optionally with stirring.

According to any aspect or embodiment of the process, the mixture may be maintained for: about 2 to about 20 hours, about 6 to about 20 hours, about 8 to about 20 hours, about 10 to about 20 hours, and preferably about 16 hours. The mixture may be cooled, preferably to a temperature of about 15° C. to about 35° C., about 20° C. to about 30° C., or about 25° C. The Form DL1 may be optionally isolated by any suitable method, for example, by centrifuge, by decantation or by filtration, preferably by filtration. The Form DL1 may be dried, preferably at a temperature of: about 20° C. to about 80° C., about 20° C. to about 70° C., about 25° C. to about 65° C., 25° C. to about 60° C., about 20° C. to about 60° C., or about 35° C. about 60° C., or about 40° C. to about 50° C., or at about 20° C. to about 30° C., or about 22° C. to about 27° C., or about 25° C. Preferably the drying is carried out under vacuum or under reduced pressure. When the drying is carried out under reduced pressure, a pressure of: about 1 or about 200 mbar, about 1 to about 100 mbar, about 1 to about 50 mbar, and particularly about 5 to about 40 mbar or more particularly, about 20 mbar, may be used.

According to any aspect of the disclosed process, Delgocitinib Form DL1 may be prepared by a process comprising: (i) stirring a suspension of Delgocitinib (preferably Form alpha, Form DL3 or Form DL7 as described in any aspect or embodiment of the disclosure), at a temperature of: about 40° C. to about 65° C. (preferably about 50° C. to about 65° C., about 55° C. to about 65° C., or about 60° C.) for: about 2 to about 20 hours (preferably about 6 to about 20 hours, about 8 to about 20 hours, about 10 to about 20 hours, or preferably about 16 hours); and (ii) cooling the mixture to a temperature of: about 15° C. to about 35° C. (preferably: about 20° C. to about 30° C., or about 25° C.); (iii) isolating the Delgocitinib Form DL1 (preferably by filtration); and optionally (iv) drying the Delgocitinib Form DL1. Preferably the drying is carried out under vacuum or under reduced pressure. The drying may be carried out at a temperature of about 20° C. to about 80° C., about 20° C. to about 70° C., about 25° C. to about 65° C., about 25° C. to about 60° C., or about 35° C. about 60° C., or about 40° C. to about 50° C.), or at about 35° C. about 60° C.; or at about 20° C. to about 30° C., or about 22° C. to about 27° C., or about 25° C. Preferably, the drying is conducted for a suitable period of time, particularly for: about 30 minutes to about 3 hours, about 30 minutes to about 2 hours, or about 1 hour. When the drying is carried out under reduced pressure, a pressure of: about 1 or about 200 mbar, about 1 to about 100 mbar, about 1 to about 50 mbar, and particularly about 5 to about 40 mbar or more particularly, about 20 mbar, may be used.

The above described processes for preparing Form DL1 of Delgocitinib may further comprise combining the Form DL1 of Delgocitinib with at least one pharmaceutically acceptable excipient to prepare a pharmaceutical formulation or dosage form.

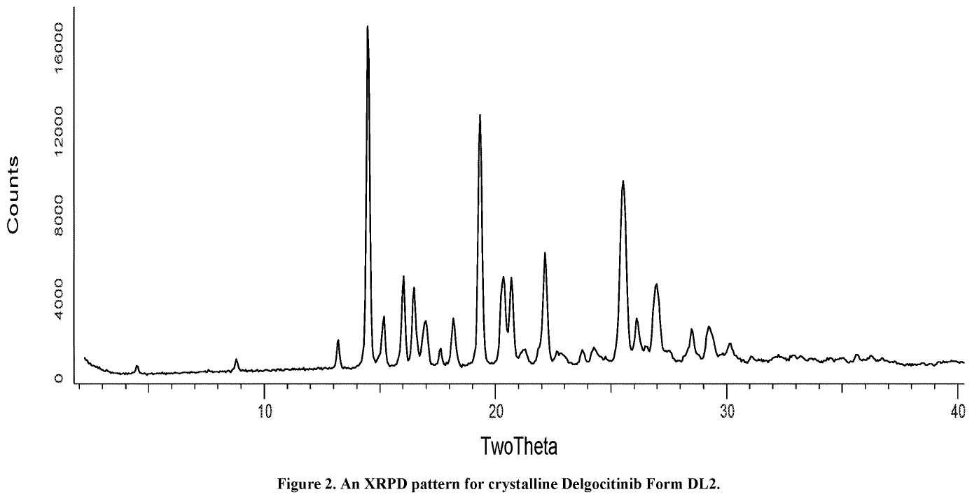

The present disclosure comprises a crystalline form of Delgocitinib designated as Form DL2. The crystalline Form DL2 of Delgocitinib can be characterized by data selected from one or more of the following: an XRPD pattern having peaks at 4.5, 8.8, 14.5, 18.2, 28.5 and 29.3 degrees 2-theta±0.2 degrees 2-theta; an XRPD pattern substantially as depicted in FIG. 2; or combinations of these data.

Crystalline Delgocitinib Form DL2 may be further characterized by data selected from one or more of the following: an XRPD pattern having peaks at 4.5, 8.8, 14.5, 18.2, 28.5 and 29.3 degrees two theta±0.2 degrees two theta; and also having one, two or three additional peaks selected from 19.3, 22.2 and 25.5 degrees two theta±0.2 degrees two theta; or combinations of these data.

Crystalline Delgocitinib Form DL2 my alternatively be characterized by XRPD pattern having peaks at 4.5, 8.8, 14.5, 18.2, 19.3, 22.2, 25.5, 28.5 and 29.3 degrees two theta±0.2 degrees two theta.

Crystalline Delgocitinib Form DL2 as described in any aspect or embodiment disclosed herein may be additionally characterized by an XRPD pattern having an absence of peaks at any one, two, three, four, or five of: 3.6 to 4.0 degrees two theta±0.2 degrees two theta; 5.0 to 8.2 degrees two theta±0.2 degrees two theta; 9.2 to 12.6 degrees two theta±0.2 degrees two theta; 13.7 to 14.1 degrees two theta±0.2 degrees two theta; and 18.7 to 18.8 degrees two theta±0.2 degrees two theta.

Crystalline Delgocitinib Form DL2 may be characterized by each of the above characteristics alone/or by all possible combinations, e.g. by XRPD pattern having peaks at 4.5, 8.8, 14.5, 18.2, 28.5 and 29.3 degrees two theta±0.2 degrees two theta; an XRPD pattern as depicted in FIG. 2, and combinations thereof.

Crystalline Delgocitinib Form DL2 may be characterized as an anhydrous form.

Delgocitinib Form DL2 as described in any aspect or embodiment of the present disclosure can be prepared by a process including suspending Delgocitinib (preferably amorphous Delgocitinib) in a solvent selected from: dimethyl formamide, isopropyl acetate, tert-butanol and cyclohexane, and particularly cyclohexane, optionally isolating the Delgocitinib Form DL2, and optionally drying.

According to any aspect or embodiment of the process, the solvent may be used in an amount of: about 20 to about 200 vol, about 50 to about 150 vol, about 70 to about 130 vol, about 90 to about 110 vol, or about 100 vol of solvent is used. The mixture may be heated to a temperature of: about 30° C. to about 70° C., about 40° C. to about 65° C., about 50° C. to about 65° C., about 55° C. to about 65° C., or about 60° C., optionally with stirring.

According to any aspect or embodiment of the process, the mixture may be heated for about 6 to about 48 hours, about 10 to about 48 hours, about 20 to about 30 hours, and in embodiments about 24 hours. The mixture may be cooled, preferably to a temperature of about 15° C. to about 35° C., about 20° C. to about 30° C., or about 25° C. The Form DL2 may be optionally isolated by any suitable method, for example by centrifuge, by decantation or by filtration, preferably by filtration. The Form DL2 may be dried, preferably at a temperature of about: 20° C. to about 80° C., about 20° C. to about 70° C., about 25° C. to about 65° C., about 35° C. about 60° C., or about 40° C. to about 50° C.; or at about 20° C. to about 30° C., or about 22° C. to about 27° C., or about 25° C. Preferably the drying is carried out under vacuum or under reduced pressure. When the drying is carried out under reduced pressure, a pressure of: about 1 or about 200 mbar, about 1 to about 100 mbar, about 1 to about 50 mbar, and particularly about 5 to about 40 mbar or more particularly, about 20 mbar, may be used.

In any aspect or embodiment of the process, Delgocitinib Form DL2 may be prepared by a process comprising: (i) stirring a suspension of Delgocitinib (preferably amorphous form), in cyclohexane, preferably at a temperature of: about 30° C. to about 70° C. (preferably: about 40° C. to about 65° C., about 50° C. to about 65° C., about 55° C. to about 65° C., or about 60° C.) for: 6 to about 48 hours (preferably about 10 to about 48 hours, about 20 to about 30 hours, and in embodiments about 24 hours); and (ii) cooling the mixture to a temperature of: about 15° C. to about 35° C. (preferably: about 20° C. to about 30° C., or about 25° C.); (iii) isolating the Delgocitinib Form DL2 (preferably by filtration); and optionally (iv) drying the Delgocitinib Form DL2 (preferably under reduced pressure at a temperature of about 20° C. to about 80° C., about 20° C. to about 70° C., about 25° C. to about 65° C., or about 35° C. about 60° C., about 40° C. to about 50° C., or about 20° C. to about 50° C.).

The above described processes for preparing Form DL2 of Delgocitinib may further comprise combining the Form DL2 of Delgocitinib with at least one pharmaceutically acceptable excipient to prepare a pharmaceutical formulation or dosage form.

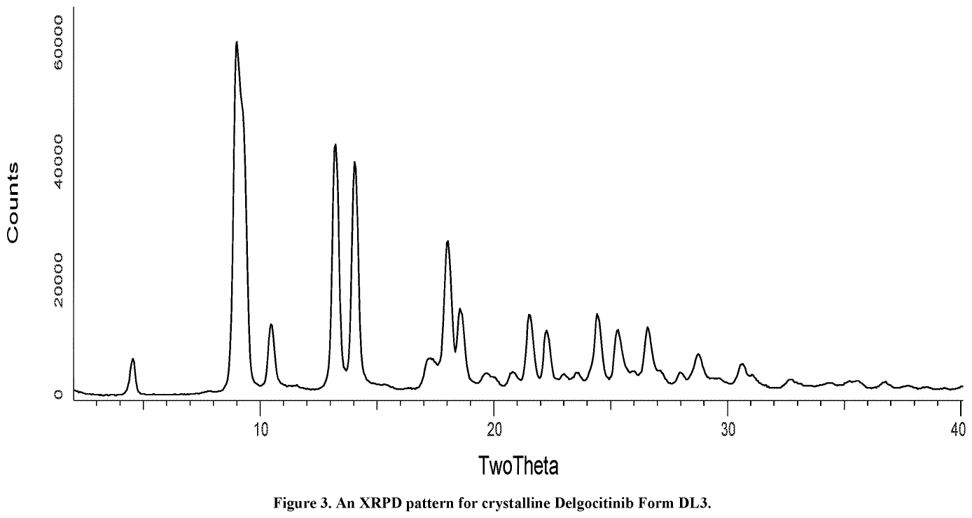

The present disclosure comprises a crystalline Delgocitinib designated as Form DL3. Delgocitinib crystalline Form DL3 can be characterized by data selected from one or more of the following: an XRPD pattern having peaks at 9.1, 18.6, 21.6, 26.6 and 28.8 degrees 2-theta±0.2 degrees 2-theta; an XRPD pattern substantially as depicted in FIG. 3; or combinations of these data.

Crystalline Delgocitinib Form DL3 may be further characterized by data selected from one or more of the following: an XRPD pattern having peaks at 9.1, 18.6, 21.6, 26.6 and 28.8 degrees two theta±0.2 degrees two theta; and also having one, two, three or four additional peaks selected from 14.1, 22.3, 24.4 and 25.3 degrees two theta±0.2 degrees two theta; or combinations of these data.

Crystalline Delgocitinib Form DL3 may alternatively be characterized by XRPD pattern having peaks at 9.1, 14.1, 18.6, 21.6, 22.3, 24.4, 25.3, 26.6 and 28.8 degrees two theta±0.2 degrees two theta.

Crystalline Delgocitinib Form DL3 as described in any aspect or embodiment disclosed herein may be additionally characterized by an XRPD pattern having an absence of peaks at any one, two or three of: 2.8 to 3.8 degrees two theta±0.2 degrees two theta; 5.2 to 7.7 degrees two theta±0.2 degrees two theta; and 12.0 to 12.6 degrees two theta±0.2 degrees two theta.

Crystalline Delgocitinib Form DL3 may be characterized by each of the above characteristics alone/or by all possible combinations, e.g. by XRPD pattern having peaks at 9.1, 18.6, 21.6, 26.6 and 28.8 degrees two theta±0.2 degrees two theta and an XRPD pattern as depicted in FIG. 3, and combinations thereof.

Crystalline Delgocitinib Form DL3 may be characterized as an anhydrous form.

The above Delgocitinib Form DL3 as described in any aspect or embodiment of the present disclosure can be prepared by exposing Delgocitinib (preferably Form DL1 or Form DL3, and more preferably Form DL1) to a temperature of: about 110° C. to about 140° C., about 115° C. to about 125° C., or about 120° C., for a suitable time to prepare Form DL3. Preferably, according to any embodiment of the process, the Delgocitinib may be exposed to this temperature for a period of: about 2 to about 10 hours, about 3 to about 8 hours, about 3 to 6 hours, or 4 hours. Preferably, in any embodiment of this process, Form DL3 may be prepared by exposing Delgocitinib (preferably Form DL1) to a temperature of: about 115° C. to about 125° C., or about 120° C., for a period of: about 3 to about 8 hours, about 3 to 6 hours, in an air tray dryer. The Form DL3 may be cooled to room temperature.

The above described processes for preparing Form DL3 of Delgocitinib may further comprise combining the Form DL3 of Delgocitinib with at least one pharmaceutically acceptable excipient to prepare a pharmaceutical formulation or dosage form.

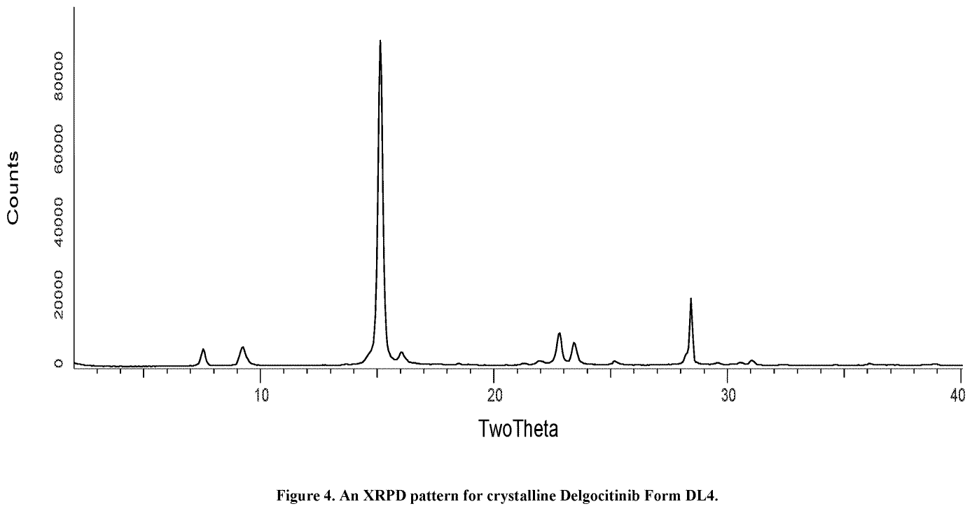

The present disclosure comprises a crystalline Delgocitinib designated as Form DL4. Delgocitinib crystalline Form DL4 can be characterized by data selected from one or more of the following: an XRPD pattern having peaks at 7.6, 15.1, 16.1, 22.8 and 23.4 degrees 2-theta±0.2 degrees 2-theta; an XRPD pattern substantially as depicted in FIG. 4; or combinations of these data.

Crystalline Delgocitinib Form DL4 may be further characterized by data selected from one or more of the following: an XRPD pattern having peaks at 7.6, 15.1, 16.1, 22.8 and 23.4 degrees two theta±0.2 degrees two theta; and also having one, two or three additional peaks selected from 9.3, 25.2 and 28.3 degrees two theta±0.2 degrees two theta; or combinations of these data.

Crystalline Delgocitinib Form DL4 may alternatively be characterized by XRPD pattern having peaks at 7.6, 9.3, 15.1, 16.1, 22.8, 25.2, 23.4 and 28.3 degrees two theta±0.2 degrees two theta.

Crystalline Delgocitinib Form DL4 as described in any aspect or embodiment disclosed herein may be additionally characterized by an XRPD pattern having an absence of peaks at any one, two, three, four, or five of: 2.5 to 6.8 degrees two theta #0.2 degrees two theta; 8.2 to 8.7 degrees two theta±0.2 degrees two theta; 10.0 to 13.2 degrees two theta±0.2 degrees two theta; and 26.0 to 27.7 degrees two theta±0.2 degrees two theta.

Crystalline Delgocitinib Form DL4 may be characterized by each of the above characteristics alone/or by all possible combinations, e.g. by XRPD pattern having peaks at 7.6, 15.1, 16.1, 22.8 and 23.4 degrees two theta±0.2 degrees two theta and an XRPD pattern as depicted in FIG. 4, and combinations thereof.

Crystalline Delgocitinib Form DL4 may be characterized as a hydrate form.

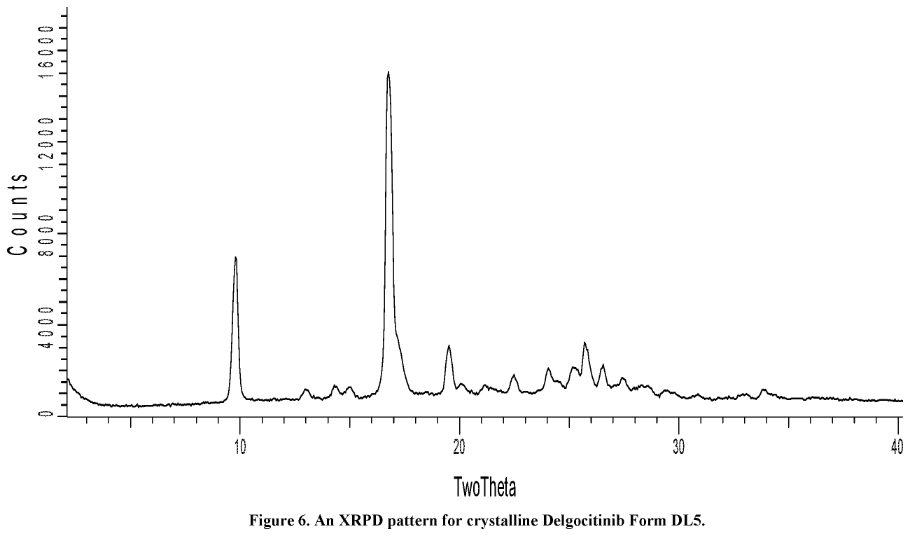

The present disclosure comprises a crystalline Delgocitinib designated as Form DL5. Delgocitinib crystalline Form DL5 can be characterized by data selected from one or more of the following: an XRPD pattern having peaks at 9.8, 16.8, 22.4, 25.7 and 26.5 degrees 2-theta±0.2 degrees 2-theta; an XRPD pattern substantially as depicted in FIG. 6; or combinations of these data.

Crystalline Delgocitinib Form DL5 may be further characterized by data selected from one or more of the following: an XRPD pattern having peaks at 9.8, 16.8, 22.4, 25.7 and 26.5 degrees two theta±0.2 degrees two theta; and also having one, two, three or four additional peaks selected from 13.0, 14.3, 19.5 and 25.2 degrees two theta±0.2 degrees two theta; or combinations of these data.

Crystalline Delgocitinib Form DL5 may alternatively be characterized by XRPD pattern having peaks at 9.8, 13.0, 14.3, 16.8, 19.5, 22.4, 25.2, 25.7 and 26.5 degrees two theta±0.2 degrees two theta.

Crystalline Delgocitinib Form DL5 as described in any aspect or embodiment disclosed herein may be additionally characterized by an XRPD pattern having an absence of peaks at one or both of: 4.0 to 9.0 degrees two theta±0.2 degrees two theta; and 10.5 to 12.2 degrees two theta±0.2 degrees two theta.

Crystalline Delgocitinib Form DL5 may be characterized by each of the above characteristics alone/or by all possible combinations, e.g. by XRPD pattern having peaks at 9.8, 16.8, 22.4, 25.7 and 26.5 degrees two theta±0.2 degrees two theta and an XRPD pattern as depicted in FIG. 6, and combinations thereof.

Crystalline Delgocitinib Form DL5 may be characterized as a hydrate form.

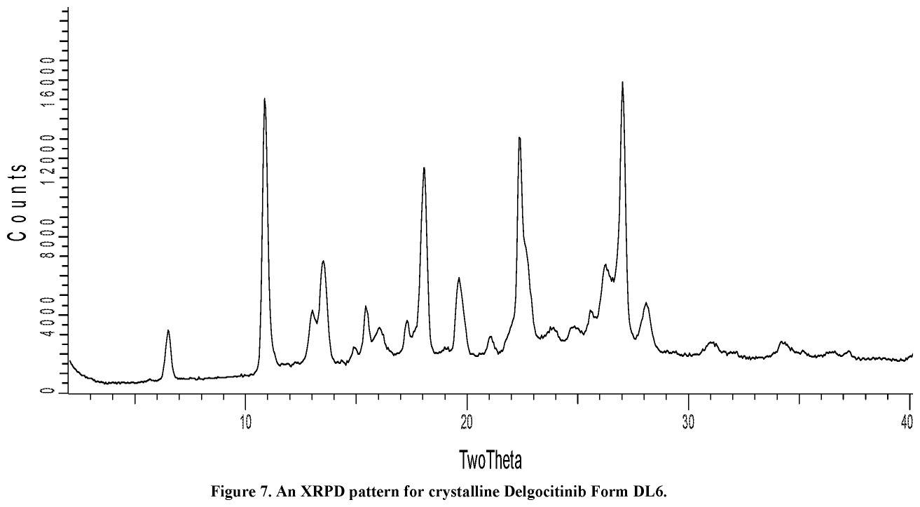

The present disclosure comprises a crystalline Delgocitinib designated as Form DL6. Delgocitinib crystalline Form DL6 can be characterized by data selected from one or more of the following: an XRPD pattern having peaks at 6.5, 10.9, 13.5, 15.4 and 18.0 degrees 2-theta±0.2 degrees 2-theta; an XRPD pattern substantially as depicted in FIG. 7; or combinations of these data.

Crystalline Delgocitinib Form DL6 may be further characterized by data selected from one or more of the following: an XRPD pattern having peaks at 6.5, 10.9, 13.5, 15.4 and 18.0 degrees two theta±0.2 degrees two theta; and also having one, two or three additional peaks selected from 19.6, 22.4 and 27.0 degrees two theta±0.2 degrees two theta; or combinations of these data.

Crystalline Delgocitinib Form DL6 may alternatively be characterized by XRPD pattern having peaks at 6.5, 10.9, 13.5, 15.4, 18.0, 19.6, 22.4 and 27.0 degrees two theta±0.2 degrees two theta.

Crystalline Delgocitinib Form DL6 as described in any aspect or embodiment disclosed herein may be additionally characterized by an XRPD pattern having an absence of peaks at one or both of: 3.5 to 5.2 degrees two theta±0.2 degrees two theta; and 7.2 to 10.0 degrees two theta±0.2 degrees two theta.

Crystalline Delgocitinib Form DL6 may be characterized by each of the above characteristics alone/or by all possible combinations, e.g. by XRPD pattern having peaks at 6.5, 10.9, 13.5, 15.4 and 18.0 degrees two theta±0.2 degrees two theta and an XRPD pattern as depicted in FIG. 7, and combinations thereof.

Crystalline Delgocitinib Form DL6 may be characterized as a hydrate form.

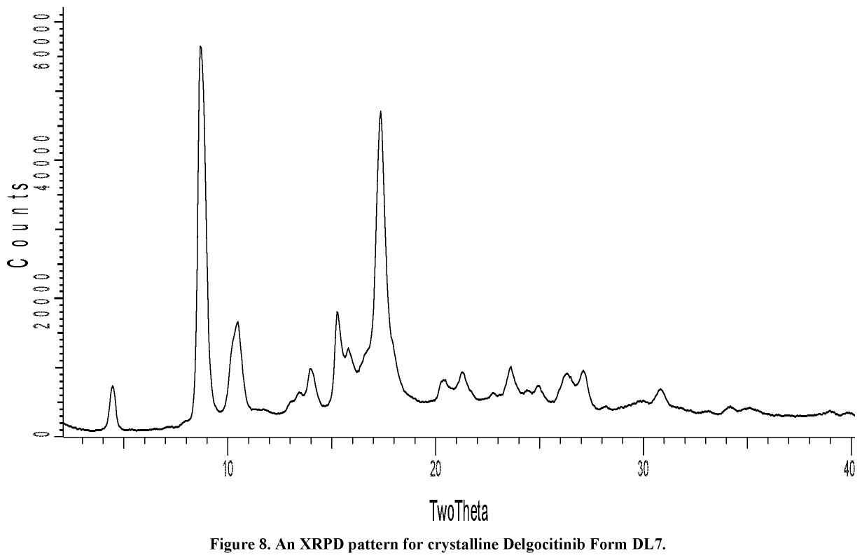

The present disclosure comprises a crystalline Delgocitinib designated as Form DL7. Delgocitinib crystalline Form DL7 can be characterized by data selected from one or more of the following: an XRPD pattern having peaks at 4.5, 8.8, 10.5, 14.0, 15.3 and 15.8 degrees 2-theta±0.2 degrees 2-theta; an XRPD pattern substantially as depicted in FIG. 8; or combinations of these data.

Crystalline Delgocitinib Form DL7 may be further characterized by data selected from one or more of the following: an XRPD pattern having peaks at 4.5, 8.8, 10.5, 14.0, 15.3 and 15.8 degrees two theta±0.2 degrees two theta; and also having one, two or three additional peaks selected from 17.4, 23.6 and 27.1 degrees two theta±0.2 degrees two theta; or combinations of these data.

Crystalline Delgocitinib Form DL7 may alternatively be characterized by XRPD pattern having peaks at 4.5, 8.8, 10.5, 14.0, 15.3, 15.8, 17.4, 23.6 and 27.1 degrees two theta #0.2 degrees two theta.

Crystalline Delgocitinib Form DL7 as described in any aspect or embodiment disclosed herein may be additionally characterized by an XRPD pattern having an absence of peaks at 5.2 to 7.0 degrees two theta±0.2 degrees two theta.

Crystalline Delgocitinib Form DL7 may be characterized by each of the above characteristics alone/or by all possible combinations, e.g. by XRPD pattern having peaks at 4.5, 8.8, 10.5, 14.0, 15.3 and 15.8 degrees two theta±0.2 degrees two theta and an XRPD pattern as depicted in FIG. 8, and combinations thereof.

Crystalline Delgocitinib Form DL7 may be characterized as a hydrate form.

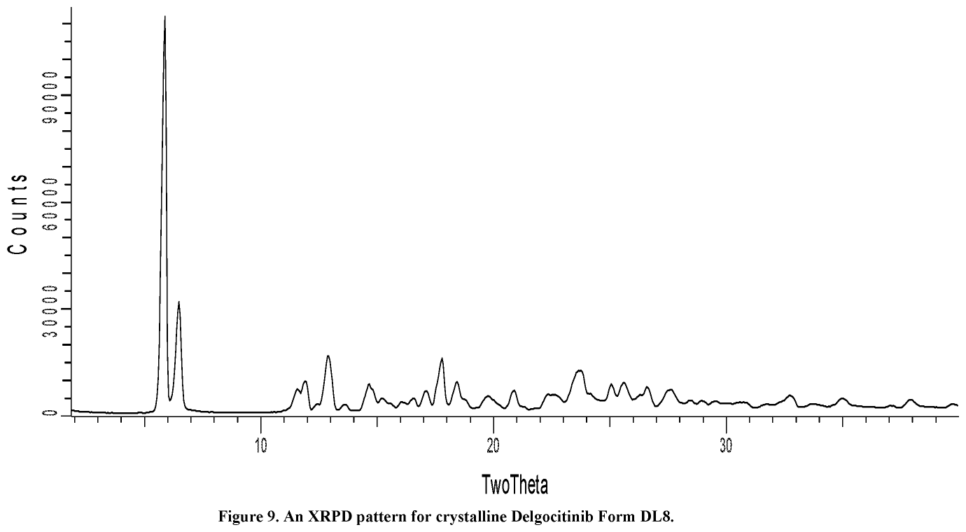

The present disclosure comprises a crystalline Delgocitinib designated as Form DL8. Delgocitinib crystalline Form DL8 can be characterized by data selected from one or more of the following: an XRPD pattern having peaks at 5.9, 6.5, 12.9, 17.8 and 25.7 degrees 2-theta±0.2 degrees 2-theta; an XRPD pattern substantially as depicted in FIG. 9; or combinations of these data.

Crystalline Delgocitinib Form DL8 may be further characterized by data selected from one or more of the following: an XRPD pattern having peaks at 5.9, 6.5, 12.9, 17.8 and 25.7 degrees two theta±0.2 degrees two theta; and also having one, two, three or four additional peaks selected from 12.0, 14.8, 18.5 and 20.9 degrees two theta±0.2 degrees two theta; or combinations of these data.

Crystalline Delgocitinib Form DL8 may alternatively be characterized by XRPD pattern having peaks at 5.9, 6.5, 12.0, 12.9, 14.8, 17.8, 18.5, 20.9 and 25.7 degrees two theta±0.2 degrees two theta.

Crystalline Delgocitinib Form DL8 as described in any aspect or embodiment disclosed herein may be additionally characterized by an XRPD pattern having an absence of peaks at one or both of: 2.7 to 4.6 degrees two theta±0.2 degrees two theta; and 7.6 to 10.8 degrees two theta±0.2 degrees two theta.

Crystalline Delgocitinib Form DL8 may be characterized by each of the above characteristics alone/or by all possible combinations, e.g. by XRPD pattern having peaks at 5.9, 6.5, 12.9, 17.8 and 25.7 degrees two theta±0.2 degrees two theta and an XRPD pattern as depicted in FIG. 9, and combinations thereof.

Crystalline Delgocitinib Form DL8 may be characterized as acetone solvate.

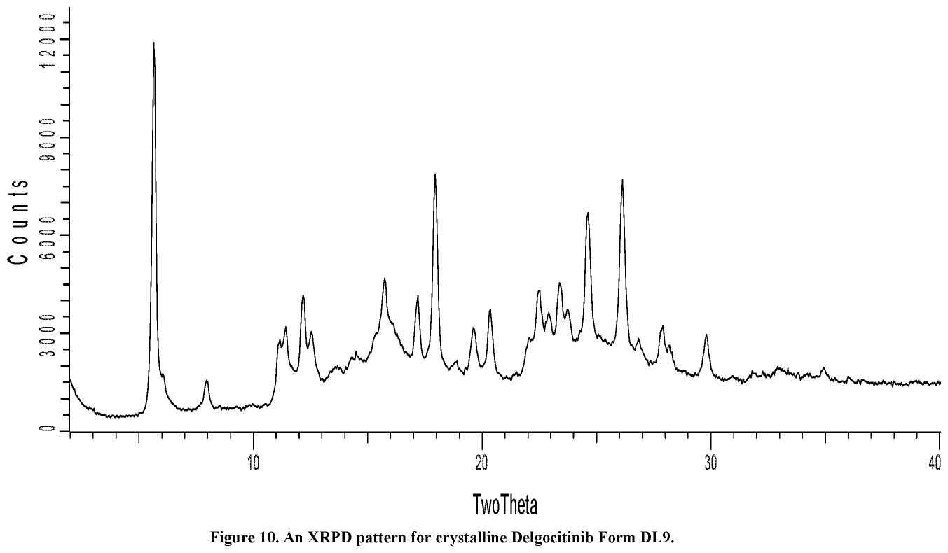

The present disclosure comprises a crystalline Delgocitinib designated as Form DL9. Delgocitinib crystalline Form DL9 can be characterized by data selected from one or more of the following: an XRPD pattern having peaks at 5.7, 17.2, 18.0, 24.6 and 26.1 degrees 2-theta±0.2 degrees 2-theta; an XRPD pattern substantially as depicted in FIG. 10; or combinations of these data.

Crystalline Delgocitinib Form DL9 may be further characterized by data selected from one or more of the following: an XRPD pattern having peaks at 5.7, 17.2, 18.0, 24.6 and 26.1 degrees two theta±0.2 degrees two theta; and also having one, two, three or four additional peaks selected from 15.8, 19.6, 20.4 and 29.8 degrees two theta±0.2 degrees two theta; or combinations of these data.

Crystalline Delgocitinib Form DL9 may alternatively be characterized by XRPD pattern having peaks at 5.7, 15.8, 17.2, 18.0, 19.6, 20.4, 24.6, 26.1 and 29.8 degrees two theta±0.2 degrees two theta.

Crystalline Delgocitinib Form DL9 as described in any aspect or embodiment disclosed herein may be additionally characterized by an XRPD pattern having an absence of peaks at one or both of: 3.5 to 4.8 degrees two theta±0.2 degrees two theta; and 7.0 to 7.5 degrees two theta±0.2 degrees two theta.

Crystalline Delgocitinib Form DL9 may be characterized by each of the above characteristics alone/or by all possible combinations, e.g. by XRPD pattern having peaks at 5.7, 17.2, 18.0, 24.6 and 26.1 degrees two theta±0.2 degrees two theta and an XRPD pattern as depicted in FIG. 10, and combinations thereof.

Crystalline Delgocitinib Form DL9 may be characterized as isopropyl alcohol solvate, preferably a hemi isopropyl alcohol solvate.

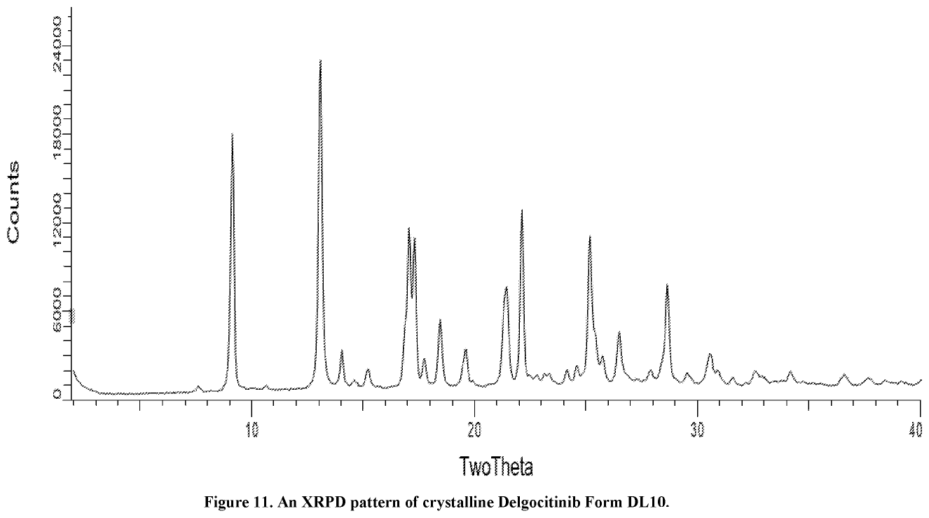

The present disclosure comprises a crystalline Delgocitinib designated as Form DL10. Delgocitinib crystalline Form DL10 can be characterized by data selected from one or more of the following: an XRPD pattern having peaks at 9.1, 13.1, 18.4, 25.1 and 28.6 degrees 2-theta±0.2 degrees 2-theta and absence of peaks at 4.5, 10.2, 11.4, 11.8, 12.7, 18.0, 20.3, 21.8, 23.6 degrees 2-theta±0.2 degrees 2-theta; an XRPD pattern substantially as depicted in FIG. 11; or combinations of these data.

Crystalline Delgocitinib Form DL10 may be further characterized by data selected from one or more of the following: an XRPD pattern having peaks at 9.1, 13.1, 18.4, 25.1 and 28.6 degrees 2-theta±0.2 degrees 2-theta and absence of peaks at 4.5, 10.2, 11.4, 11.8, 12.7, 18.0, 20.3, 21.8, 23.6 degrees 2-theta±0.2 degrees 2-theta; and also having one, two or three additional peaks selected from 19.6, 21.4 and 26.4 degrees two theta±0.2 degrees two theta; or combinations of these data.

Crystalline Delgocitinib Form DL10 may alternatively be characterized by XRPD pattern having peaks at 9.1, 13.1, 18.4, 19.6, 21.4, 25.1, 26.4 and 28.6 degrees 2-theta±0.2 degrees 2-theta and absence of peaks at 4.5, 10.2, 11.4, 11.8, 12.7, 18.0, 20.3, 21.8, 23.6 degrees 2-theta±0.2 degrees 2-theta degrees.

Crystalline Delgocitinib Form DL10 as described in any aspect or embodiment disclosed herein may be additionally characterized by an XRPD pattern having an absence of peaks at any one, two, three, or four of: at 3.5 to 7.0 degrees 2-theta±0.2 degrees 2-theta; 8.0 to 8.6 degrees 2-theta±0.2 degrees 2-theta; 11.2 to 12.4 degrees 2-theta±0.2 degrees 2-theta

Crystalline Delgocitinib Form DL10 may be characterized by each of the above characteristics alone/or by all possible combinations, e.g. by XRPD pattern having peaks at 9.1, 13.1, 18.4, 25.1 and 28.6 degrees 2-theta±0.2 degrees 2-theta and absence of peaks at 4.5, 10.2, 11.4, 11.8, 12.7, 18.0, 20.3, 21.8, 23.6 degrees 2-theta±0.2 degrees 2-theta degrees two theta±0.2 degrees two theta and an XRPD pattern as depicted in FIG. 11, and combinations thereof.

Crystalline Delgocitinib Form DL10 may be characterized as anhydrous form.

Delgocitinib Form DL10 as described in any aspect or embodiment of the present disclosure can be prepared by a process comprising a solvent/antisolvent crystallization. In any aspect or embodiment of the process, Delgocitinib Form DL10 may be prepared by crystallization of Delgocitinib from a mixture of dimethylformamide (DMF) and isobutyl acetate, optionally isolating and optionally drying. Preferably, the process may comprise combining a mixture of Delgocitinib Form DL10 in DMF with isobutyl acetate, and crystallising Delgocitinib Form DL10 from the mixture.

In any aspect or embodiment of the process, DMF may be used in an amount of about 1.5 vol to about 5 vol, about 2 to about 4 vol relative to Delgocitinib, or about 2.6 vol. Isobutyl acetate may be used in an amount of about 30 vol to about 100 vol, about 40 vol to about 80 vol and preferably about 50 vol relative to Delgocitinib. In any aspect or embodiment of the process, the volume ratio of DMF to isobutyl acetate may be: about 1:5 to about 1:30, about 1:10 to about 1:25, or about 1:19 or about 1:20.

In any aspect or embodiment of the process for preparing Delgocitinib Form DL10, isobutyl acetate may be combined with a mixture of Delgocitinib in DMF, preferably wherein the mixture of Delgocitinib in DMF is heated to a temperature of: about 40° C. to about 80° C., about 50° C. to about 70° C., about 55° C. to about 65° C., or about 60° C., preferably with stirring.

In any aspect or embodiment, the process may comprise addition of the isobutyl acetate to the solution of Delgocitinib, or, preferably, the Delgocitinib solution is added to isobutyl acetate. In any embodiment of the process, a solution of Delgocitinib in DMF, preferably at a temperature of: about 40° C. to about 80° C., about 50° C. to about 70° C., about 55° C. to about 65° C., or about 60° C., is added to isobutyl acetate. Preferably, the isobutyl acetate is at a temperature of: about −10° C. to about 10° C., about −5° C. to about 5° C., about-2° C. to about 5° C., about-2° C. to about 2° C., or about 0° C. The addition may be carried out over a period of about 1 to about 30 minutes, about 1 to about 20 minutes, about 2 to about 10 or about 3 to about 8 minutes, or about 5 minutes.

According to any embodiment of the above processes for preparing Form DL10, the reaction mixture comprising Delgocitinib, DMF and isobutyl acetate, may be maintained at a temperature of: about −10° C. to about 10° C., about −5° C. to about 5° C., about −2° C. to about 5° C., about −2° C. to about 2° C., or about 0° C. The mixture may be maintained at this temperature for: about 1 to about 30 hours, about 10 to about 25 hours, about 15 to about 20 hours, or about 18 hours, preferably with stirring.

In any embodiment of the above processes for preparing Form DL10, the Delgocitinib Form DL10 may be isolated. The isolation may be carried out by any suitable means, such as by centrifuge, decantation, or by filtration, preferably by filtration. Preferably, the filtration is carried out at a temperature of about 10° C. to about 40° C., preferably about 20° C. to about 30° C., or about room temperature. Following the isolation, the product may be washed, and optionally dried. Drying may be done by nitrogen or air or under vacuum. Drying may be performed at a temperature of about 20° C. to about 50° C., or about 20° C. to about 40° C., or about 20° C. to about 30° C. or about 25° C. The drying may be carried out for a sufficient time for Form DL10, preferably for: about 5 min to 2 hours, in embodiment 10-15 minutes. When the drying is carried out under vacuum, a pressure of: about 1 or about 200 mbar, about 1 to about 100 mbar, about 1 to about 50 mbar, and particularly about 5 to about 40 mbar or more particularly, about 20 mbar, may be used.

Delgocitinib Form DL10 as described in any aspect or embodiment of the disclosure may be prepared by a process comprising: (i) combining a solution of Delgocitinib in DMF (preferably at a temperature of: about 30° C. to about 70° C. (preferably: about 40° C. to about 65° C., about 50° C. to about 65° C., about 55° C. to about 65° C., or about 60° C.) with isobutyl acetate (preferably wherein the isobutyl acetate is at a temperature of: about −10° C. to about 10° C., about −5° C. to about 5° C., about-2° C. to about 5° C., about-2° C. to about 2° C., or about 0° C.), (ii) maintaining the mixture for: about 2 to about 24 hours (preferably about 6 to about 22 hours, about 8 to about 20 hours, about 10 to about 20 hours, or about 18 hours), preferably at a temperature of: about −10° C. to about 10° C., about −5° C. to about 5° C., about-2° C. to about 5° C., about-2° C. to about 2° C., or about 0° C.; and (ii) cooling the mixture to a temperature of: about 15° C. to about 35° C. (preferably at a temperature of: about 20° C. to about 30° C., or about 25° C.); (iii) isolating the Delgocitinib Form DL10 (preferably by filtration); and optionally (iv) drying the Delgocitinib Form DL10 (preferably under reduced pressure at a temperature of about 20° C. to about 80° C., about 20° C. to about 70° C., about 25° C. to about 65° C., or about 35° C. about 60° C., or about 40° C. to about 50° C.); or at about 20° C. to about 30° C., or about 22° C. to about 27° C., or about 25° C.

When the drying is carried out under reduced pressure, a pressure of: about 1 or about 200 mbar, about 1 to about 100 mbar, about 1 to about 50 mbar, and particularly about 5 to about 40 mbar or more particularly, about 20 mbar, may be used.

The above-described processes for preparing Form DL10 of Delgocitinib may further comprise combining the Form DL10 of Delgocitinib with at least one pharmaceutically acceptable excipient to prepare a pharmaceutical formulation or dosage form.

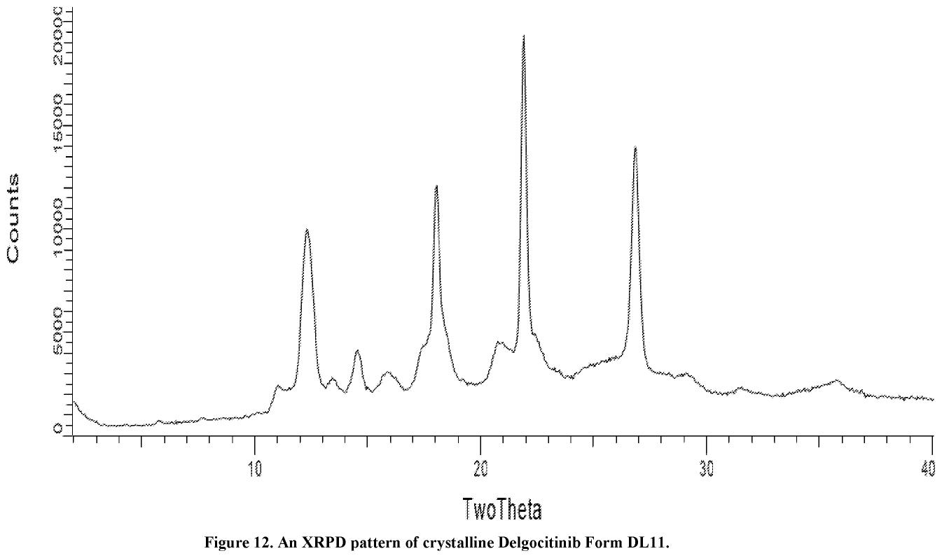

The present disclosure comprises a crystalline form of Delgocitinib designated as Form DL11. The crystalline Form DL11 of Delgocitinib can be characterized by data selected from one or more of the following: an XRPD pattern having peaks at 12.4, 14.6, 18.1, 21.9, 26.9 degrees 2-theta±0.2 degrees 2-theta and absence of peaks at 4.5, 8.8, 10.2, 11.4, 11.8, 15.6, 16.5, 19.3, 20.3, 23.8 and 26.0 degrees 2-theta±0.2 degrees 2-theta; an XRPD pattern substantially as depicted in FIG. 12; or combinations of these data.

Crystalline Delgocitinib Form DL11 may be further characterized by data selected from one or more of the following: an XRPD pattern having peaks at 12.4, 14.6, 18.1, 21.9, 26.9 degrees 2-theta±0.2 degrees 2-theta and absence of peaks at 4.5, 8.8, 10.2, 11.4, 11.8, 15.6, 16.5, 19.3, 20.3, 23.8 and 26.0 degrees 2-theta±0.2 degrees 2-theta; and also having one, two or three additional peaks selected from 11.0, 21.0 and 29.1 degrees two theta±0.2 degrees two theta; or combinations of these data.

Crystalline Delgocitinib Form DL11 my alternatively be characterized by XRPD pattern having peaks at 11.0, 12.4, 14.6, 18.1, 21.0, 21.9, 26.9 and 29.1 degrees 2-theta±0.2 degrees 2-theta and absence of peaks at 4.5, 8.8, 10.2, 11.4, 11.8, 15.6, 16.5, 19.3, 20.3, 23.8 and 26.0 degrees 2-theta±0.2 degrees 2-theta degrees two theta±0.2 degrees two theta.

Crystalline Delgocitinib Form DL11 as described in any aspect or embodiment disclosed herein may be additionally characterized by an XRPD pattern having an absence of peaks at any one, two, three, or four of: 3.5 to 5.2 degrees 2-theta±0.2 degrees 2-theta; 3.5 to 5.2 degrees 2-theta±0.2 degrees 2-theta; at 6.5 to 7.7 degrees 2-theta±0.2 degrees 2-theta; and 8.2 to 10.1 degrees 2-theta±0.2 degrees 2-theta.

Crystalline Delgocitinib Form DL11 may be characterized by each of the above characteristics alone/or by all possible combinations, e.g. by XRPD pattern having peaks at 12.4, 14.6, 18.1, 21.9, 26.9 degrees 2-theta±0.2 degrees 2-theta and absence of peaks at 4.5, 8.8, 10.2, 11.4, 11.8, 15.6, 16.5, 19.3, 20.3, 23.8 and 26.0 degrees 2-theta±0.2 degrees 2-theta; an XRPD pattern as depicted in FIG. 12, and combinations thereof.

Crystalline Delgocitinib Form DL11 may be characterized as an anhydrous form.

The above Delgocitinib Form DL11 as described in any aspect or embodiment of the present disclosure can be prepared by exposing Delgocitinib (preferably Form DL6 as described in any aspect or embodiment herein) to temperature of about 95° C. to about 105° C., or about 100° C., for a suitable time to prepare Form DL11. The Delgocitinib may be exposed to this temperature for a period of: about 10 to about 60 minutes, about 20 to about 40 minutes; or about 30 minutes. Preferably, in any embodiment of this process, Form DL11 may be prepared by a process comprising exposing Delgocitinib (preferably Form DL6) to a temperature of: about 95° C. to about 105° C., or about 100° C., for a period of: about 10 to about 60 minutes, about 20 to about 40 minutes; or about 30 minutes, in an air tray dryer. The Form DL11 may be cooled to room temperature.

The above described processes for preparing Form DL11 of Delgocitinib may further comprise combining the Form DL11 of Delgocitinib with at least one pharmaceutically acceptable excipient to prepare a pharmaceutical formulation or dosage form.

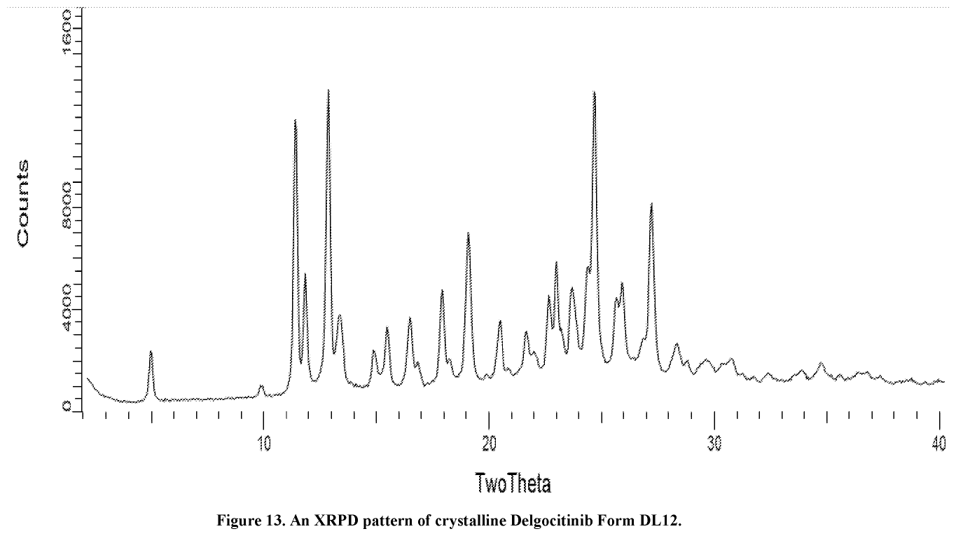

The present disclosure comprises a crystalline Delgocitinib designated as Form DL12. Delgocitinib crystalline Form DL12 can be characterized by data selected from one or more of the following: an XRPD pattern having peaks at 4.9, 11.4, 12.9, 19.1, and 24.7 degrees 2-theta±0.2 degrees 2-theta; an XRPD pattern substantially as depicted in FIG. 13; or combinations of these data.

Crystalline Delgocitinib Form DL12 may be further characterized by data selected from one or more of the following: an XRPD pattern having peaks at 4.9, 11.4, 12.9, 19.1, and 24.7 degrees two theta±0.2 degrees two theta; and also having one, two, three or four additional peaks selected from 11.8, 16.5, 17.9 and 27.2 degrees two theta±0.2 degrees two theta; or combinations of these data.

Crystalline Delgocitinib Form DL12 may alternatively be characterized by XRPD pattern having peaks at 4.9, 11.4, 11.8, 12.9, 16.5, 17.9, 19.1, 24.7 and 27.2 degrees two theta±0.2 degrees two theta.

Crystalline Delgocitinib Form DL12 as described in any aspect or embodiment disclosed herein may be additionally characterized by an XRPD pattern having an absence of peaks at one or both of: 3.8 to 4.5 degrees two theta±0.2 degrees two theta; and 5.5 to 9.0 degrees two theta±0.2 degrees two theta.

Crystalline Delgocitinib Form DL12 may be characterized by each of the above characteristics alone/or by all possible combinations, e.g. by XRPD pattern having peaks at 4.9, 11.4, 12.9, 19.1, and 24.7 degrees two theta±0.2 degrees two theta and an XRPD pattern as depicted in FIG. 13, and combinations thereof.

Crystalline Delgocitinib Form DL12 may be characterized as benzyl solvate, preferably mono benzyl solvate form.

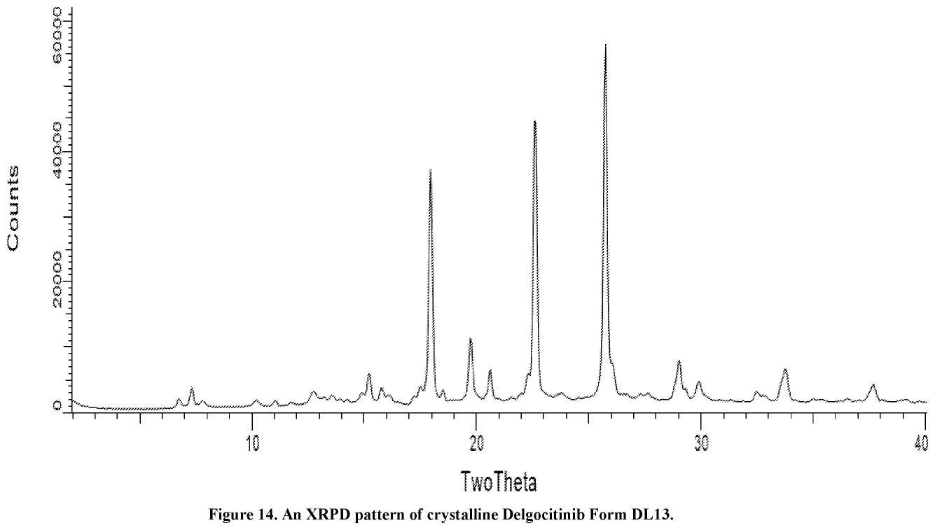

The present disclosure comprises a crystalline Delgocitinib designated as Form DL13. Delgocitinib crystalline Form DL13 can be characterized by data selected from one or more of the following: an XRPD pattern having peaks at 15.2, 18.0, 19.8, 22.7 and 25.8 degrees 2-theta±0.2 degrees 2-theta; an XRPD pattern substantially as depicted in FIG. 14; or combinations of these data.

Crystalline Delgocitinib Form DL13 may be further characterized by data selected from one or more of the following: an XRPD pattern having peaks at 15.2, 18.0, 19.8, 22.7 and 25.8 degrees two theta±0.2 degrees two theta; and also having one, two, three or four additional peaks selected from 7.4, 20.7, 29.1 and 33.8 degrees two theta±0.2 degrees two theta; or combinations of these data.

Crystalline Delgocitinib Form DL13 may alternatively be characterized by XRPD pattern having peaks at 7.4, 15.2, 18.0, 19.8, 20.7, 22.7, 25.8, 29.1 and 33.8 degrees two theta±0.2 degrees two theta.

Crystalline Delgocitinib Form DL13 as described in any aspect or embodiment disclosed herein may be additionally characterized by an XRPD pattern having an absence of peaks at one or both of: 3.0 to 6.0 degrees two theta±0.2 degrees two theta; and 8.5 to 9.5 degrees two theta±0.2 degrees two theta.

Crystalline Delgocitinib Form DL13 may be characterized by each of the above characteristics alone/or by all possible combinations, e.g. by XRPD pattern having peaks at 15.2, 18.0, 19.8, 22.7 and 25.8 degrees two theta±0.2 degrees two theta and an XRPD pattern as depicted in FIG. 14, and combinations thereof.

Crystalline Delgocitinib Form DL13 may be characterized as ethanol solvate.

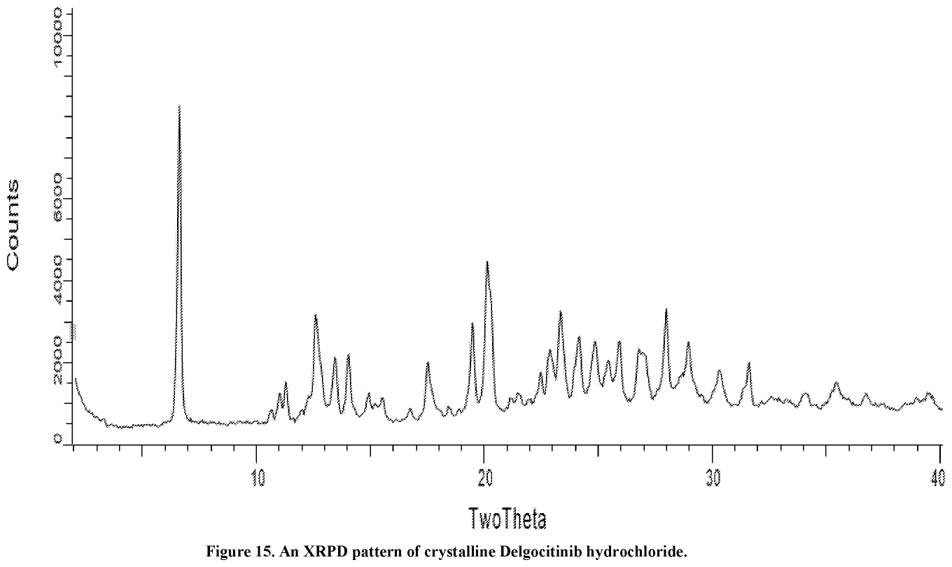

The present disclosure comprises a crystalline Delgocitinib hydrochloride. Crystalline Delgocitinib hydrochloride can be characterized by data selected from one or more of the following: an XRPD pattern having peaks at 6.6, 12.6, 19.5, 20.1 and 28.0 degrees 2-theta±0.2 degrees 2-theta; an XRPD pattern substantially as depicted in FIG. 15; or combinations of these data.

Crystalline Delgocitinib hydrochloride may be further characterized by data selected from one or more of the following: an XRPD pattern having peaks at 6.6, 12.6, 19.5, 20.1 and 28.0 degrees two theta±0.2 degrees two theta; and also having one, two, three or four additional peaks selected from 14.0, 23.4, 24.2 and 24.9 degrees two theta±0.2 degrees two theta; or combinations of these data.

Crystalline Delgocitinib hydrochloride may alternatively be characterized by XRPD pattern having peaks at 6.6, 12.6, 14.0, 19.5, 20.1, 23.4, 24.2, 24.9 and 28.0 degrees two theta±0.2 degrees two theta.

Crystalline Delgocitinib hydrochloride as described in any aspect or embodiment disclosed herein may be additionally characterized by an XRPD pattern having an absence of peaks at one or both of: 4.0 to 5.5 degrees two theta±0.2 degrees two theta; and 7.2 to 8.5 degrees two theta±0.2 degrees two theta.

Crystalline Delgocitinib hydrochloride may be characterized by each of the above characteristics alone/or by all possible combinations, e.g. by XRPD pattern having peaks at 6.6, 12.6, 19.5, 20.1 and 28.0 degrees two theta±0.2 degrees two theta and an XRPD pattern as depicted in FIG. 15, and combinations thereof.

In another embodiment of the present disclosure, crystalline Delgocitinib Forms DL1-DL13 and crystalline Delgocitinib hydrochloride are polymorphically and chemically pure.

The present disclosure also relates to a crystalline forms of Delgocitinib Forms DL1-DL13 and crystalline Delgocitinib hydrochloride which are obtainable by any process as described herein. The said process can include the process set out in the examples herein below.

The present disclosure also relates to a pharmaceutical composition comprising crystalline Delgocitinib Forms DL1-DL13 and crystalline Delgocitinib hydrochloride as described herein, or crystalline Delgocitinib Forms DL1-DL13 and crystalline Delgocitinib hydrochloride which is obtainable by any process as described herein below.

The invention further comprises a process for preparing a pharmaceutical composition comprising crystalline Delgocitinib Forms DL1-DL13 and crystalline Delgocitinib hydrochloride, wherein the process comprises combining crystalline Delgocitinib Forms DL1-DL13 and crystalline Delgocitinib hydrochloride with at least one pharmaceutically acceptable excipient. The invention further comprises a process for preparing crystalline Delgocitinib Forms DL1-DL13 and crystalline Delgocitinib hydrochloride as described herein below and further comprising combining crystalline Delgocitinib Forms DL1-DL13 and crystalline Delgocitinib hydrochloride with at least one pharmaceutically acceptable excipient.

The present disclosure also provides for processes for the preparation of the solid state form of Delgocitinib and Delgocitinib hydrochloride. The said process can include the process set out in the examples herein below.

In another aspect, the present disclosure encompasses the above described solid state form of Delgocitinib and crystalline Delgocitinib hydrochloride for use in the preparation of pharmaceutical compositions and/or formulations, preferably for use in medicine, preferably for treating atopic dermatitis.

In another aspect, the present disclosure encompasses the use of the above described solid state form of Delgocitinib and crystalline Delgocitinib hydrochloride for the preparation of pharmaceutical compositions and/or formulations, preferably for use in medicine, preferably for treating atopic dermatitis.

In yet another embodiment, the present disclosure encompasses pharmaceutical compositions comprising the solid state form of Delgocitinib and crystalline Delgocitinib hydrochloride.

In specific embodiment, the present disclosure encompasses pharmaceutical formulation comprising the solid state form of Delgocitinib and crystalline Delgocitinib hydrochloride, and at least one pharmaceutically acceptable excipient.

The present disclosure further encompasses processes to prepare said pharmaceutical formulations of Delgocitinib and crystalline Delgocitinib hydrochloride, comprising combining the above described solid state form of Delgocitinib and crystalline Delgocitinib hydrochloride, or pharmaceutical compositions comprising them, and at least one pharmaceutically acceptable excipient.

The present disclosure comprises processes for preparing the above mentioned pharmaceutical compositions. The processes comprise combining the above crystalline polymorph of Delgocitinib and crystalline Delgocitinib hydrochloride, of the present disclosure with at least one pharmaceutically acceptable excipient.

Pharmaceutical formulations of the present invention contain the crystalline polymorph of Delgocitinib of the present invention, particularly crystalline Delgocitinib Forms DL1-DL13 and crystalline Delgocitinib hydrochloride. In addition to the active ingredient, the pharmaceutical formulations of the present invention can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Delgocitinib and Delgocitinib hydrochloride and any other solid excipients are dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present invention, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present invention is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present invention can be a capsule containing the composition, preferably a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, which causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, but they are not subjected to a final tableting step.

The solid state forms of Delgocitinib and Delgocitinib hydrochloride as defined herein as well as the pharmaceutical compositions or formulations comprising them can be used as medicaments, particularly for treating atopic dermatitis, comprising administering a therapeutically effective amount of the solid state form of the present disclosure, or at least one of the above pharmaceutical compositions or formulations, to a subject suffering from atopic dermatitis, or otherwise in need of the treatment.

The present disclosure also provides the uses of the solid state form of Delgocitinib and Delgocitinib hydrochloride of the present disclosure, or at least one of the above pharmaceutical compositions or formulations, for the manufacture of medicaments for treating atopic dermatitis.

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### Analytical Methods

Powder X-ray Diffraction was performed on an X-Ray powder diffractometer Bruker D8 Advance; CuK radiation (λ=1.5418 Å); Lynx eye detector; laboratory temperature 22-25° C.; PMMA specimen holder ring. Prior to analysis, the samples were gently ground by means of mortar and pestle in order to obtain a fine powder. The ground sample was adjusted into a cavity of the sample holder and the surface of the sample was smoothed by means of a cover glass.

Measurement parameters: Scan range: 2-40 degrees 2-theta; Scan mode: continuous; Step size: 0.05 degrees; Time per step: 0.5 s; Sample spin: 30 rpm; Sample holder: PMMA specimen holder ring with silicon low background.

All X-Ray Powder Diffraction peak values are calibrated with regard to standard silicon spiking in the sample.

### EXAMPLES

Delgocitinib can be obtained by any procedure described in the literature, for example using the syntheses procedure reported in U.S. Pat. No. 8,609,647 or 10,822,354.

Delgocitinib Form alpha used in example below can be produced according to procedure described in U.S. Pat. No. 11,339,181 and according to procedure 19 below.

### Example 1: Preparation of Delgocitinib Form DL1

Delgocitinib (Form alpha, 5.0 grams) and 150 ml water were heated to 60° C. and maintained for about 16 hours under stirring. The reaction mass was cooled down to about 25° C., filtered, dried under vacuum for 1 hour and analyzed by XRPD to obtain Delgocitinib designated Form DL1 (FIG. 1).

### Example 2. Preparation of Delgocitinib Form DL2

Delgocitinib Form DL1 (0.01 grams) was dried at 190° C. for 2 minutes in Thermogravimetric analyzer (heating to 190° C. at 10 deg/min and keeping stable at 190° C. for 2 min). The sample was cooled down to about 25° C. and analyzed by XRPD to obtain Delgocitinib designated Form DL2 (FIG. 2).

### Example 3. Preparation of Delgocitinib Form DL3

Delgocitinib Form DL4 (0.1 grams) was dried at 120° C. for 4 hours in Air tray drier (ATD). The sample was cooled down to about 25° C. and analyzed by XRPD to obtain Delgocitinib designated Form DL3 (FIG. 3).

### Example 4. Preparation of Delgocitinib Form DL4

Delgocitinib (0.05 grams) and Buffer solution (4.5 pH) (2 mL) were heated to 80° C. The obtained clear solution was cool down to −20° C. in about 1 hour and maintained for 15 hours at this temperature under stirring. The reaction mass was heated to 25° C., filtered, dried under vacuum for 20-25 minutes and analyzed by XRPD to obtain Delgocitinib designated Form DL4 (FIG. 4).

### Example 5. Preparation of Amorphous Delgocitinib

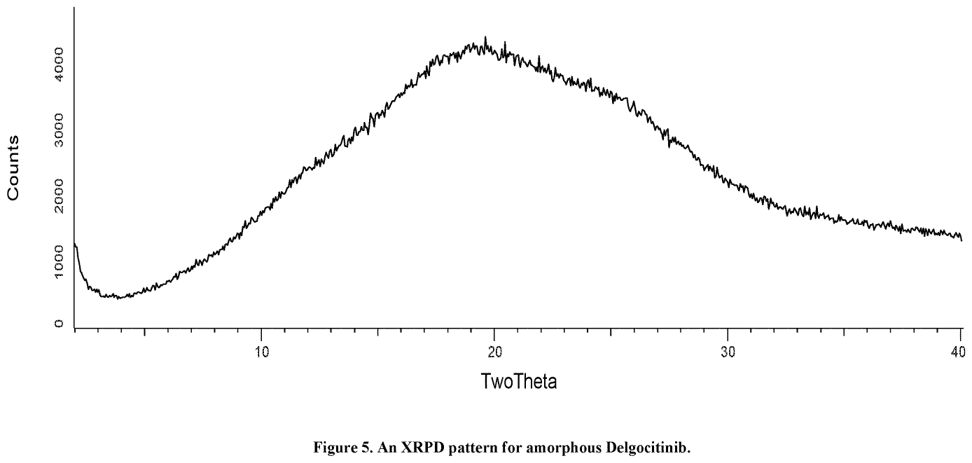

Delgocitinib (0.8 grams) was dissolved in Acetone (80 mL) at 55° C. The clear solution was subjected to distillation under high vacuum (below 50 mbar) on rotary evaporator at 55° C. for about 30-45 minutes. The obtained solid was isolated and analyzed by XRPD to obtain amorphous Delgocitinib (FIG. 5).

### Example 6. Preparation of Delgocitinib Form DL4

Amorphous Delgocitinib (0.1 grams) and water (4 ml) were kept at 25° C. for about 4-5 hours under stirring. The reaction mass was filtered, dried under vacuum for 20-25 minutes and analyzed by XRPD to contain Form DL4.

### Example 7. Preparation of Delgocitinib Form DL5

Delgocitinib Form DL4 (0.1 grams) was dried at 100° C. for 30 minutes in Air tray drier (ATD). The sample was cooled to about 25° C. and the dried solid was designated Form DL5 by XRPD (FIG. 6).

### Example 8. Preparation of Delgocitinib Form DL6

Amorphous Delgocitinib (0.1 grams) was exposed to 100% relative humidity (RH) at 25° C. for 7 days in in Humidity chamber (ATD). The analyzed solid was designated Form DL6 by XRPD (FIG. 7).

### Example 9. Preparation of Delgocitinib Form DL7

Delgocitinib Form DL1 (0.1 grams) was dried at 100° C. for 30 minutes in Air tray drier (ATD). The dried sample was cooled to about 25° C. and analyzed by XRPD to be designated Form DL7 (FIG. 8).

### Example 10. Preparation of Delgocitinib Form DL8

Delgocitinib (1.0 gram) was dissolved in Acetone (120 mL) at 50° C. The clear solution was subjected to distillation under high vacuum (around 300 mbar) on rotary evaporator at 50° C. for about 1 hour. The solid was isolated and was analyzed by XRPD to be designated Form DL8 (FIG. 9).

### Example 11. Preparation of Delgocitinib Form DL9

Amorphous Delgocitinib (0.03 grams) was grinded with Isopropyl alcohol (0.05 ml) using mortar and pestle for 2 minutes. The obtained solid was analyzed by XRPD and designated as Form DL9 (FIG. 10).

### Example 12. Preparation of Delgocitinib Form DL2

Amorphous Delgocitinib (0.05 grams) and Cyclohexane (5 ml) were heated to 60° C. for 1 day under stirring. The reaction mass was cooled down to about 25° C., filtered and dried under vacuum for 20-25 minutes. The obtained solid was designated as Form DL2 by XRPD.

### Example 13. Preparation of Delgocitinib Form DL3

Delgocitinib Form DL1 (2 grams) was dried at 120° C. for 4 hours in Air tray drier (ATD). The sample was cooled down to about 25° C. The solid was analyzed by XRPD and designated as Form DL3.

### Example 14: Preparation of Crystalline Delgocitinib Form DL10

Delgocitinib (1 gram) was dissolved in DMF (2.6 ml) at 60° C. to obtain clear solution. The clear solution was added into precooled Isobutyl acetate (50 ml) at 0° C. in a reactor. The reaction mixture was maintained for about 18 hours at about 0° C. under stirring. The hot reaction mixture was filtered, the isolated solid was dried under vacuum for 10-15 minutes and analyzed by XRPD to obtain Delgocitinib designated Form DL10 (FIG. 1).

### Example 15. Preparation of Crystalline Delgocitinib Form DL11

Delgocitinib Form DL6 (0.1 grams) was dried at 100° C. for 30 minutes in Air tray drier (ATD). The sample was cooled to about 25° C. and analyzed by XRPD to obtain Delgocitinib designated Form DL11 (FIG. 12).

### Example 16. Preparation of Crystalline Delgocitinib Form DL12

Delgocitinib (0.03 grams) was dissolved in Benzyl alcohol (0.2 ml) at 60° C. to obtain a clear solution. The clear solution was added into precooled Tert-butyl acetate (3 ml) at 0° C. in a reactor. The reaction mixture was maintained under stirring for about 18 hours at about 0° C., filtered to isolate a solid dried under vacuum for 10-15 minutes and analyzed by XRPD to obtain Delgocitinib designated Form DL12 (FIG. 13).

### Example 17. Preparation of Crystalline Delgocitinib Form DL13

Delgocitinib (Form a, 0.05 grams) was dissolved in Ethanol (4 ml) at 60° C. to obtain clear solution. The clear solution was added into preheated (about 80° C.) n-Heptane (15 ml) at 80° C. in a reactor. Reaction mixture was maintained under stirring at about 80° C. for about 1 hour. The hot reaction mixture was filtrated and the isolated solid was dried under vacuum for 10-15 minutes and designated Delgocitinib Form DL13 by XRPD (FIG. 14).

### Example 18. Preparation of Crystalline Delgocitinib Hydrochloride

Delgocitinib (Form a 500 mg) was added to 10 ml Isopropyl acetate at 25° C. IPA.HCl (0.425 ml) was added and maintained under stirring for about 1 day at about 25° C. The reaction mass was filtered and the isolated solid was dried under vacuum for 20-25 minutes and analyzed by XRPD to obtain crystalline Delgocitinib hydrochloride (FIG. 15).

### Example 19. Preparation of Crystalline Form Alpha

Delgocitinib (0.03 grams) was dissolved in 1-Butanol (1 ml) at 60° C. to obtain a clear solution. The clear solution was added into precooled n-Decane (5 ml) at 0° C. and maintained for about 18 hours under stirring at about 0° C. The reaction mixture was filtered and the isolated solid was dried under vacuum for 10-15 minutes and designated Form Alpha by XRPD (FIG. 16).

### Example 20. Preparation of Delgocitinib Form DL4

A buffer solution at pH 4.5 was prepared according to the US Pharmacopoeia standard (0.02 M Sodium acetate solution and 2N Acetic acid solution): Weigh 27.20 grams of Sodium Acetate Trihydrate into a one liter volumetric flask. Add 800 mL of deionized water. Mix and dissolve. Bring the pH down to 4.5 with glacial acetic acid. Finally adjust the volume to one liter with deionized water to obtain 1000 mL of solution having a pH of 4.50±0.05.

A mixture of Delgocitinib (0.05 grams) and the buffer solution (2 mL) were heated to 80° C. The resulting solution was cool to −20° C. over about 1 hour and maintained for 15 hours at this temperature under stirring. The reaction mixture was allowed to warm to 25° C., and the mixture was filtered. The product was dried under vacuum at room temperature for 20-25 minutes. XRPD analysis confirmed the product to be Delgocitinib designated Form DL4.

### Example 21: Preparation of Crystalline Delgocitinib Form DL10

Delgocitinib (1 gram) was dissolved in DMF (2.6 ml) at 60° C. to obtain a clear solution. The solution was added into precooled isobutyl acetate (50 ml) at 0° C. The reaction mixture was maintained for about 18 hours at about 0° C. under stirring. The mixture was allowed to cool, and the solid was isolated by filtration, and dried at room temperature under vacuum for 10-15 minutes. XRPD confirmed the presence of Form DL10.

### Example 22. Preparation of Crystalline Delgocitinib Hydrochloride

Delgocitinib Form a (0.5 grams) was added to isopropyl acetate (10 ml) at 25° C. A solution of HCl (g) in isopropanol (18%, 0.425 ml) was added. The reaction mixture was stirred for about 1 day at about 25° C. The solid was isolated by filtration, and the product, crystalline Delgocitinib hydrochloride, was dried at room temperature under vacuum for 20-25 minutes.

### Example 23. Stability Studies

### Storage Stability at Different Relative Humidities

Samples of crystalline forms of Delgocitinib according to the disclosure, were subjected to conditions of different relative humidities at ambient temperature. XRPD analysis was performed on the samples after 7 days. The results are shown in Table 1 below:

TABLE 1

| | |
|---|---|
| Starting | XRPD after storage at different relative |
| Delgocitinib | humidities for 7 days |

| form | 20% | 40% | 60% | 80% |
|---|---|---|---|---|
| DL1 | DL1 | DL1 | DL1 | DL1 |
| DL2 | DL2 | DL2 | DL2 | DL2 |
| DL3 | DL3 | DL3 | DL3 | DL3 |
| DL10 | DL10 | DL10 | DL10 | DL10 |
| DL11 | DL11 | DL11 | DL11 | DL11 |

These results demonstrate that Forms DL1, DL2, DL3, DL10 and DL11 are stable after exposure to high and low relative humidity conditions for at least 7 days.

Samples of Delgocitinib polymorph forms according to the disclosure were subjected to conditions of different relative humidities at different temperatures. Forms DL1 was kept in low-density polyethylene bag (twist and tied) covered by triple laminated bag (heat and sealed). Forms DL2, DL3, DL10 and DL11 were kept in low-density polyethylene bag (twist and tied) and covered by triple laminated bag (heat and sealed), while 2 gram silica gel sachet was added in between the two bags. XRPD analysis was performed on the samples after 1, 2, 3 and 6 months of storage, and the results are shown in Tables 2 and 3 below:

TABLE 2

| | |
|---|---|
| Starting | XRPD after storage at 25° C. at 60% RH |
| Delgocitinib | for 1, 2, 3 and 6 months |

| form | 1 month | 2 months | 3 months | 6 months |
|---|---|---|---|---|
| DL1 | DL1 | DL1 | DL1 | DL1 |
| DL2 | DL2 | DL2 | DL2 | DL2 |
| DL3 | DL3 | DL3 | DL3 | DL3 |
| DL10 | DL10 | DL10 | DL10 | DL10 |
| DL11 | DL11 | DL11 | DL11 | DL11 |

TABLE 3

| | |
|---|---|
| Starting | XRPD after storage at 40° C. at 75% RH |
| Delgocitinib | for 1, 2, 3 and 6 months |

| form | 1 month | 2 months | 3 months | 6 months |
|---|---|---|---|---|
| DL1 | DL1 | DL1 | DL1 | DL1 |
| DL2 | DL2 | DL2 | DL2 | DL2 |
| DL3 | DL3 | DL3 | DL3 | DL3 |
| DL10 | DL10 | DL10 | DL10 | DL10 |
| DL11 | DL11 | DL11 | DL11 | DL11 |

The results demonstrate that Forms DL1, DL2, DL3, DL10, and DL11 are highly storage stable.

### Grinding Experiments

Samples of Delgocitinib forms according to the disclosure were subjected to strong grinding. In these experiments, about 20 mg of the sample was placed in a mortar and ground with a pestle for 2 minutes. XRPD analysis was performed on each sample after the grinding experiment (Table 4):

TABLE 4

| Starting | |
|---|---|
| Delgocitinib form | XRPD after grinding |
| DL1 | DL1 |
| DL2 | DL2 |
| DL3 | DL3 |
| DL10 | DL10 |
| DL11 | DL11 |

The results demonstrate that Delgocitinib Forms DL1, DL2, DL3, DL10, and D11, are resistant to polymorphic changes and is highly suitable for preparing pharmaceutical formulations.

### Stability to Compression

Samples of Delgocitinib were subjected to pressures of 2 tons (Atlas® Autopress hydraulic press, set to 2 tons). XRPD analysis was carried out on the samples after 2 minutes. The results are shown in Table 5 below:

TABLE 5

| | XRPD after compression |
|---|---|
| Starting | force (2 tons for 2 |
| Delgocitinib form | minutes |
| DL1 | DL1 |
| DL2 | DL2 |
| DL3 | DL3 |
| DL10 | DL10 |
| DL11 | DL11 |

Accordingly, Forms DL1, DL2, DL3, DL10, and DL11 OF Delgocitinib are stable under high pressure conditions, making these forms highly suitable for pharmaceutical processing.

Further aspects and embodiments of the present disclosure are set out in the following numbered clauses:

### CLAUSES

1. Crystalline Delgocitinib designated as Form DL2, which is characterized by data selected from: an XRPD pattern having peaks at 4.5, 8.8, 14.5, 18.2, 28.5 and 29.3 degrees 2-theta±0.2 degrees 2-theta; or an XRPD pattern substantially as depicted in FIG. 2.
2. Crystalline Delgocitinib Form DL2 according to Clause 1, which is characterized by an XRPD pattern having peaks at 4.5, 8.8, 14.5, 18.2, 28.5 and 29.3 degrees two theta±0.2 degrees two theta; and also having one, two or three additional peaks selected from 19.3, 22.2 and 25.5 degrees two theta±0.2 degrees two theta.
3. Crystalline Delgocitinib Form DL2 according to Clause 1 or Clause 2, which is characterized by an XRPD pattern having peaks at 4.5, 8.8, 14.5, 18.2, 19.3, 22.2, 25.5, 28.5 and 29.3 degrees two theta±0.2 degrees two theta.
4. Crystalline Delgocitinib Form DL2 according to any of Clauses 1, 2 or 3, which is characterized by an XRPD pattern having an absence of peaks at 3.6 to 4.0 degrees two theta±0.2 degrees two theta.
5. Crystalline Delgocitinib Form DL2 according to any of Clauses 1, 2, 3, or 4, which is characterized by an XRPD pattern having an absence of peaks at 5.0 to 8.2 degrees two theta±0.2 degrees two theta.
6. Crystalline Delgocitinib Form DL2 according to any of Clauses 1, 2, 3, 4, or 5, which is characterized by an XRPD pattern having an absence of peaks at 9.2 to 12.6 degrees two theta±0.2 degrees two theta.
7. Crystalline Delgocitinib Form DL2 according to any of Clauses 1, 2, 3, 4, 5, or 6, which is characterized by an XRPD pattern having an absence of peaks at 13.7 to 14.1 degrees two theta±0.2 degrees two theta.
8. Crystalline Delgocitinib Form DL2 according to any of Clauses 1, 2, 3, 4, 5, 6, or 7, which is characterized by an XRPD pattern having an absence of peaks at 18.7 to 18.8 degrees two theta±0.2 degrees two theta.
9. Crystalline Delgocitinib Form DL2 according to any of Clauses 1, 2, 3, 4, 5, 6, 7, or 8, which is an anhydrous form.
10. Crystalline Delgocitinib designated as Form DL1, which is characterized by data selected from: an XRPD pattern having peaks at 9.5, 12.6, 14.3, 16.8 and 18.1 degrees 2-theta #0.2 degrees 2-theta; or an XRPD pattern substantially as depicted in FIG. 1.
11. Crystalline Delgocitinib Form DL1 according to Clause 10, which is characterized by data selected from one or more of the following: an XRPD pattern having peaks at 9.5, 12.6, 14.3, 16.8 and 18.1 degrees two theta±0.2 degrees two theta; and also having one, two or three additional peaks selected from 9.0, 19.1 and 27.4 degrees two theta±0.2 degrees two theta.
12. Crystalline Delgocitinib Form DL1 according to Clause 10 or Clause 11, which is characterized by an XRPD pattern having peaks at 9.0, 9.5, 12.6, 14.3, 16.8, 18.1, 19.1 and 27.4 degrees two theta±0.2 degrees two theta.
13. Crystalline Delgocitinib Form DL1 according to any of Clauses 10, 11 or 12, which is characterized by an XRPD pattern having an absence of peaks at 3.0 to 6.5 degrees two theta±0.2 degrees two theta.
14. Crystalline Delgocitinib Form DL1 according to any of Clauses 10, 11, 12, or 13, which is characterized by an XRPD pattern having an absence of peaks at 7.8 to 8.5 degrees two theta±0.2 degrees two theta.
15. Crystalline Delgocitinib Form DL1 according to any of Clauses 10, 11, 12, 13, or 14, which is characterized by an XRPD pattern having an absence of peaks at 10.0 to 10.6 degrees two theta±0.2 degrees two theta.
16. A crystalline form of Delgocitinib Form DL1 according to any of Clauses 10, 11, 12, 13, 14, or 15, which is characterized by an XRPD pattern having an absence of peaks at 11.6 to 12.0 degrees two theta±0.2 degrees two theta.
17. Crystalline Delgocitinib Form DL1 according to any of Clauses 10, 11, 12, 13, 14, 15, or 16, which is characterized by an XRPD pattern having an absence of peaks at 13.6 to 13.8 degrees two theta±0.2 degrees two theta.
18. Crystalline Delgocitinib Form DL1 according to any of Clauses 10, 11, 12, 13, 14, 15, 16, or 17, which is characterized by an XRPD pattern having an absence of peaks at 19.9 to 20.2 degrees two theta±0.2 degrees two theta.
19. Crystalline Delgocitinib Form DL1 according to any of Clauses 10, 11, 12, 13, 14, 15, 16, 17, or 18, which is characterized by an XRPD pattern having an absence of peaks at 22.9 to 23.4 degrees two theta±0.2 degrees two theta.
20. Crystalline Delgocitinib Form DL1 according to any of Clauses 10, 11, 12, 13, 14, 15, 16, 17, 18, or 19, which is a hydrate, and optionally which is a monohydrate.
21. Crystalline Delgocitinib designated as Form DL3, which is characterized by data selected from: an XRPD pattern having peaks at 9.1, 18.6, 21.6, 26.6 and 28.8 degrees 2-theta±0.2 degrees 2-theta; or an XRPD pattern substantially as depicted in FIG. 3.
22. Crystalline Delgocitinib Form DL3 according to Clause 21, which is characterized by data selected from one or more of the following: an XRPD pattern having peaks at 9.1, 18.6, 21.6, 26.6 and 28.8 degrees two theta±0.2 degrees two theta; and also having one, two, three or four additional peaks selected from 14.1, 22.3, 24.4 and 25.3 degrees two theta±0.2 degrees two theta.
23. Crystalline Delgocitinib Form DL3 according to Clause 21 or Clause 22, which is characterized by an XRPD pattern having peaks at 9.1, 14.1, 18.6, 21.6, 22.3, 24.4, 25.3, 26.6 and 28.8 degrees two theta±0.2 degrees two theta.
24. Crystalline Delgocitinib Form DL3 according to any of Clauses 21, 22 or 23, which is characterized by an XRPD pattern having an absence of peaks at 2.8 to 3.8 degrees two theta±0.2 degrees two theta.
25. Crystalline Delgocitinib Form DL3 according to any of Clauses 21, 22, 23, or 24, which is characterized by an XRPD pattern having an absence of peaks at 5.2 to 7.7 degrees two theta±0.2 degrees two theta.
26. Crystalline Delgocitinib Form DL3 according to any of Clauses 21, 22, 23, 24, or 25, which is characterized by an XRPD pattern having an absence of peaks at 12.0 to 12.6 degrees two theta±0.2 degrees two theta.
27. Crystalline Delgocitinib Form DL3 according to any of Clauses 21, 22, 23, 24, 25, or 26, which is an anhydrous form.
28. Crystalline Delgocitinib designated as Form DL10, which is characterized by data selected from:
   (i) an XRPD pattern having peaks at 9.1, 13.1, 18.4, 25.1 and 28.6 degrees 2-theta±0.2 degrees 2-theta, and absence of peaks at 4.5, 10.2, 11.4, 11.8, 12.7, 18.0, 20.3, 21.8, and 23.6 degrees 2-theta±0.2 degrees 2-theta; or
   (ii) an XRPD pattern substantially as depicted in FIG. 11.
29. Crystalline Delgocitinib Form DL10 according to Clause 28, which is characterized by data selected from one or more of the following: an XRPD pattern having peaks at 9.1, 13.1, 18.4, 25.1 and 28.6 degrees 2-theta±0.2 degrees 2-theta and absence of peaks at 4.5, 10.2, 11.4, 11.8, 12.7, 18.0, 20.3, 21.8, and 23.6 degrees 2-theta±0.2 degrees 2-theta; and also having one, two or three additional peaks selected from 19.6, 21.4 and 26.4 degrees two theta±0.2 degrees two theta.
30. Crystalline Delgocitinib Form DL10 according to Clause 28 or Clause 29, which is characterized by XRPD pattern having peaks at 9.1, 13.1, 18.4, 19.6, 21.4, 25.1, 26.4 and 28.6 degrees 2-theta±0.2 degrees 2-theta, and absence of peaks at 4.5, 10.2, 11.4, 11.8, 12.7, 18.0, 20.3, 21.8, and 23.6 degrees 2-theta±0.2 degrees 2-theta degrees.
31. Crystalline Delgocitinib Form DL10 according to any of Clauses 28, 29 or 30, which is further characterized by an XRPD pattern having an absence of peaks at 3.5 to 7.0 degrees 2-theta±0.2 degrees 2-theta.
32. Crystalline Delgocitinib designated as Form DL10, which is characterized by an XRPD pattern having peaks at 9.1, 13.1, 18.4, 25.1, and 28.6 degrees 2-theta±0.2 degrees 2-theta, and optionally an absence of peaks at 3.5 to 7.0 degrees 2-theta±0.2 degrees 2-theta.
33. Crystalline Delgocitinib Form DL10 according to any of Clauses 28, 29, 30, 31, or 32, which is characterized by an absence of peaks at 8.0 to 8.6 degrees 2-theta±0.2 degrees 2-theta.
34. Crystalline Delgocitinib Form DL10 according to any of Clauses 28, 29, 30, 31, 32, or 33, which is characterized by an absence of peaks at 11.2 to 12.4 degrees 2-theta±0.2 degrees 2-theta.
35. Crystalline Delgocitinib Form DL10 according to any of Clauses 28, 29, 30, 31, 32, 33, or 34, which is an anhydrous form.
36. Crystalline Delgocitinib designated as Form DL11, which is characterized by data selected from one or more of the following:
   (i) an XRPD pattern having peaks at 12.4, 14.6, 18.1, 21.9, 26.9 degrees 2-theta±0.2 degrees 2-theta and absence of peaks at 4.5, 8.8, 10.2, 11.4, 11.8, 15.6, 16.5, 19.3, 20.3, 23.8 and 26.0 degrees 2-theta±0.2 degrees 2-theta; or
   (ii) an XRPD pattern substantially as depicted in FIG. 12.
37. Crystalline Delgocitinib Form DL11 according to Clause 36, which is characterized by: an XRPD pattern having peaks at 12.4, 14.6, 18.1, 21.9, 26.9 degrees 2-theta #0.2 degrees 2-theta and absence of peaks at 4.5, 8.8, 10.2, 11.4, 11.8, 15.6, 16.5, 19.3, 20.3, 23.8 and 26.0 degrees 2-theta±0.2 degrees 2-theta; and also having one, two or three additional peaks selected from 11.0, 21.0 and 29.1 degrees two theta±0.2 degrees two theta.
38. Crystalline Delgocitinib Form DL11 according to Clause 36 or 37, which is characterized by an XRPD pattern having peaks at 11.0, 12.4, 14.6, 18.1, 21.0, 21.9, 26.9 and 29.1 degrees 2-theta±0.2 degrees 2-theta and absence of peaks at 4.5, 8.8, 10.2, 11.4, 11.8, 15.6, 16.5, 19.3, 20.3, 23.8 and 26.0 degrees 2-theta±0.2 degrees 2-theta degrees two theta±0.2 degrees two theta.
39. Crystalline Delgocitinib Form DL11 according to any of Clauses 36, 37 or 38, which is further characterized by an XRPD pattern having an absence of peaks at 3.5 to 5.2 degrees 2-theta±0.2 degrees 2-theta.
40. Crystalline Delgocitinib designated as Form DL11, which is characterized by an XRPD pattern having peaks at 12.4, 14.6, 18.1, 21.9, and 26.9 degrees 2-theta±0.2 degrees 2-theta, and optionally an absence of peaks at 3.5 to 5.2 degrees 2-theta±0.2 degrees 2-theta.
41. Crystalline Delgocitinib Form DL11 according to any of Clauses 36, 37, 38, 39, or 40, which is characterized by an absence of peaks at 6.5 to 7.7 degrees 2-theta±0.2 degrees 2-theta.
42. Crystalline Delgocitinib Form DL11 according to any of Clauses 36, 37, 38, 39, 40, or 41, which is characterized by an absence of peaks at 8.2 to 10.1 degrees 2-theta±0.2 degrees 2-theta.
43. A crystalline form of Delgocitinib Form DL11 according to any of Clauses 36, 37, 38, 39, 40, 41, or 42, which is an anhydrous form.
44. A crystalline product according to any of Clauses 1 to 43, which contains: no more than about 20%, no more than about 10%, no more than about 5%, no more than about 2%, no more than about 1% or about 0% of any other crystalline forms of Delgocitinib.
45. A crystalline product according to any of Clauses 1 to 44, which contains: no more than about 20%, no more than about 10%, no more than about 5%, no more than about 2%, no more than about 1% or about 0% of amorphous Delgocitinib.
46. A pharmaceutical composition comprising a crystalline product according to any of Clauses 1 to 45.
47. Use of a crystalline product according to any of Clauses 1 to 45 for the preparation of a pharmaceutical composition and/or pharmaceutical formulation.
48. A pharmaceutical formulation comprising a crystalline product according to any of Clauses 1 to 45, or a pharmaceutical composition of Clause 46, with at least one pharmaceutically acceptable excipient.
49. A process for preparing the pharmaceutical formulation according to Clause 48 comprising combining a crystalline product according to any of Clauses 1 to 45, or a pharmaceutical composition of Clause 46, with at least one pharmaceutically acceptable excipient.
50. A crystalline product according to any of Clauses 1 to 45, a pharmaceutical composition according to Clause 46, or a pharmaceutical formulation according to Clause 48, for use as a medicament.
51. A crystalline product according to any of Clauses 1 to 45, a pharmaceutical composition according to Clause 46 or a pharmaceutical formulation according to Clause 48, for use in the treatment of: atopic dermatitis, including moderate to severe atopic dermatitis; eczema, including moderate to severe chronic hand eczema and atopic eczema; discoid lupus erythematosus; alopecia areata; or psoriasis; and particularly atopic dermatitis.
52. A method of treating: atopic dermatitis, including moderate to severe atopic dermatitis; eczema, including moderate to severe chronic hand eczema or atopic eczema; discoid lupus erythematosus; alopecia areata; or psoriasis; and particularly atopic dermatitis; comprising administering a therapeutically effective amount of a crystalline product according to any of Clauses 1 to 45, a pharmaceutical composition according to Clause 46, or a pharmaceutical formulation according to Clause 48 to a subject in need of the treatment.
53. A crystalline product according to any one of Clauses 1 to 45, a pharmaceutical composition according to Clause 46, or a pharmaceutical formulation according to Clause 48, for the manufacture of a medicament for the treatment of: atopic dermatitis, including moderate to severe atopic dermatitis; eczema, including moderate to severe chronic hand eczema and atopic eczema; discoid lupus erythematosus; alopecia areata; or psoriasis; and particularly atopic dermatitis.
54. Use of a crystalline product according to any one of Clauses 1 to 45, in the preparation of another solid state form of Delgocitinib or a salt of Delgocitinib or solid state form thereof.

1. A crystalline form of Delgocitinib designated as Form DL2, which is characterized by data selected from: an X-ray powder diffraction (XRPD) pattern having peaks at 4.5, 8.8, 14.5, 18.2, 28.5 and 29.3 degrees 2-theta±0.2 degrees 2-theta; or an XRPD pattern substantially as depicted in FIG. 2.

2. The crystalline form of Delgocitinib according to claim 1, which is characterized by an XRPD pattern having peaks at 4.5, 8.8, 14.5, 18.2, 28.5 and 29.3 degrees two theta±0.2 degrees two theta; and also having one, two or three additional peaks selected from 19.3, 22.2 and 25.5 degrees two theta±0.2 degrees two theta.

3. The crystalline form of Delgocitinib according to claim 1, which is characterized by an XRPD pattern having peaks at 4.5, 8.8, 14.5, 18.2, 19.3, 22.2, 25.5, 28.5 and 29.3 degrees two theta±0.2 degrees two theta.

4. The crystalline form of Delgocitinib according to claim 1, which is an anhydrous form.

5. A crystalline form of Delgocitinib designated as Form DL1, which is characterized by data selected from: an X-ray powder diffraction (XRPD) pattern having peaks at 9.5, 12.6, 14.3, 16.8 and 18.1 degrees 2-theta±0.2 degrees 2-theta; or an XRPD pattern substantially as depicted in FIG. 1.

6. The crystalline form of Delgocitinib according to claim 5, which is characterized by data selected from one or more of the following: an XRPD pattern having peaks at 9.5, 12.6, 14.3, 16.8 and 18.1 degrees two theta±0.2 degrees two theta; and also having one, two or three additional peaks selected from 9.0, 19.1 and 27.4 degrees two theta±0.2 degrees two theta.

7. The crystalline form of Delgocitinib according to claim 5, which is characterized by an XRPD pattern having peaks at 9.0, 9.5, 12.6, 14.3, 16.8, 18.1, 19.1 and 27.4 degrees two theta±0.2 degrees two theta.

8. The crystalline form of Delgocitinib according to claim 5, which is a hydrate, and optionally which is a monohydrate.

9. A crystalline form of Delgocitinib designated as Form DL3, which is characterized by data selected from: an X-ray powder diffraction (XRPD) pattern having peaks at 9.1, 18.6, 21.6, 26.6 and 28.8 degrees 2-theta±0.2 degrees 2-theta; or an XRPD pattern substantially as depicted in FIG. 3.

10. The crystalline form of Delgocitinib according to claim 9, which is characterized by data selected from one or more of the following: an XRPD pattern having peaks at 9.1, 18.6, 21.6, 26.6 and 28.8 degrees two theta±0.2 degrees two theta; and also having one, two, three or four additional peaks selected from 14.1, 22.3, 24.4 and 25.3 degrees two theta±0.2 degrees two theta.

11. The crystalline form of Delgocitinib according to claim 9, which is characterized by an XRPD pattern having peaks at 9.1, 14.1, 18.6, 21.6, 22.3, 24.4, 25.3, 26.6 and 28.8 degrees two theta±0.2 degrees two theta.

12. The crystalline form of Delgocitinib according to claim 9, which is an anhydrous form.

13. A crystalline form of Delgocitinib designated as Form DL10, which is characterized by data selected from:
(i) an X-ray powder diffraction (XRPD) pattern having peaks at 9.1, 13.1, 18.4, 25.1 and 28.6 degrees 2-theta±0.2 degrees 2-theta, and absence of peaks at 4.5, 10.2, 11.4, 11.8, 12.7, 18.0, 20.3, 21.8, and 23.6 degrees 2-theta±0.2 degrees 2-theta; or
(ii) an XRPD pattern substantially as depicted in FIG. 11.

14. The crystalline form of Delgocitinib according to claim 13, which is characterized by data selected from one or more of the following: an XRPD pattern having peaks at 9.1, 13.1, 18.4, 25.1 and 28.6 degrees 2-theta±0.2 degrees 2-theta and absence of peaks at 4.5, 10.2, 11.4, 11.8, 12.7, 18.0, 20.3, 21.8, and 23.6 degrees 2-theta±0.2 degrees 2-theta; and also having one, two or three additional peaks selected from 19.6, 21.4 and 26.4 degrees two theta±0.2 degrees two theta.

15. The crystalline form of Delgocitinib according to claim 13, which is characterized by XRPD pattern having peaks at 9.1, 13.1, 18.4, 19.6, 21.4, 25.1, 26.4 and 28.6 degrees 2-theta±0.2 degrees 2-theta, and absence of peaks at 4.5, 10.2, 11.4, 11.8, 12.7, 18.0, 20.3, 21.8, and 23.6 degrees 2-theta±0.2 degrees 2-theta degrees.

16. The crystalline form of Delgocitinib according to claim 13, which is an anhydrous form.

17. A crystalline form of Delgocitinib designated as Form DL11, which is characterized by data selected from one or more of the following:
(i) an X-ray powder diffraction (XRPD) pattern having peaks at 12.4, 14.6, 18.1, 21.9, 26.9 degrees 2-theta±0.2 degrees 2-theta and absence of peaks at 4.5, 8.8, 10.2, 11.4, 11.8, 15.6, 16.5, 19.3, 20.3, 23.8 and 26.0 degrees 2-theta±0.2 degrees 2-theta; or
(ii) an XRPD pattern substantially as depicted in FIG. 12.

18. The crystalline form of Delgocitinib Form DL11 according to claim 17, which is characterized by: an XRPD pattern having peaks at 12.4, 14.6, 18.1, 21.9, 26.9 degrees 2-theta±0.2 degrees 2-theta and absence of peaks at 4.5, 8.8, 10.2, 11.4, 11.8, 15.6, 16.5, 19.3, 20.3, 23.8 and 26.0 degrees 2-theta±0.2 degrees 2-theta; and also having one, two or three additional peaks selected from 11.0, 21.0 and 29.1 degrees two theta±0.2 degrees two theta.

19. The crystalline form of Delgocitinib according to claim 17, which is characterized by an XRPD pattern having peaks at 11.0, 12.4, 14.6, 18.1, 21.0, 21.9, 26.9 and 29.1 degrees 2-theta±0.2 degrees 2-theta and absence of peaks at 4.5, 8.8, 10.2, 11.4, 11.8, 15.6, 16.5, 19.3, 20.3, 23.8 and 26.0 degrees 2-theta±0.2 degrees 2-theta degrees two theta±0.2 degrees two theta.

20. The crystalline form of Delgocitinib Form DL11 according to claim 17, which is an anhydrous form.

21. The crystalline form of Delgocitinib according to claim 1, which contains: no more than about 20%, no more than about 10%, no more than about 5%, no more than about 2%, no more than about 1% or about 0% of any other crystalline forms of Delgocitinib.

22. The crystalline form of Delgocitinib according to claim 1, which contains: no more than about 20%, no more than about 10%, no more than about 5%, no more than about 2%, no more than about 1% or about 0% of amorphous Delgocitinib.

23. A pharmaceutical composition comprising the crystalline form of Delgocitinib according to claim 1.

24. (canceled)

25. The pharmaceutical composition of claim 23, further comprising at least one pharmaceutically acceptable excipient.

26. A process for preparing a pharmaceutical formulation, the process comprising combining the crystalline product form of Delgocitinib according to claim 1 with at least one pharmaceutically acceptable excipient.

27. A method of treating a subject in need of treatment with a medicament, the method comprising administering to the subject the crystalline product form of Delgocitinib according to claim 1.

28. (canceled)

29. The method of treating according to claim 27, wherein the method comprises treating a disease including at least one of: atopic dermatitis, including moderate to severe atopic dermatitis; eczema, including moderate to severe chronic hand eczema or atopic eczema; discoid lupus erythematosus; alopecia areata; or psoriasis.

30. (canceled)

31. A method of preparing of another solid state form of Delgocitinib or a salt of Delgocitinib or solid state form thereof comprising using the crystalline form of Delgocitinib according to claim 1 as a starting material.
